(19) United States
(12) Patent Application Publication
(10) Pub. No.: US 2025/0215053 A1
(43) Pub. Date: Jul. 3, 2025

(54) LYSINE RICH CELL-PENETRATING PEPTIDES

(71) Applicant: OXFORD UNIVERSITY INNOVATION LIMITED, Oxford (GB)

(72) Inventors: Andrey ARZUMANOV, Headington, Oxford (Oxfordshire) (GB); Yahya JAD, Headington, Oxford (Oxfordshire) (GB); David SEOANE, Headington, Oxford (Oxfordshire) (GB); Miguel VARELA, Headington, Oxford (Oxfordshire) (GB); Matthew WOOD, Headington, Oxford (Oxfordshire) (GB)

(21) Appl. No.: 18/850,580

(22) PCT Filed: Apr. 5, 2023
(86) PCT No.: PCT/GB2023/050911

§ 371 Date: Sep. 25, 2024

(30) Foreign Application Priority Data
Apr. 8, 2022 (GB) 2205213.8

(51) Int. Cl. C07K 7/08; A61K 45/06; A61K 47/64; C07K 19/00

(52) U.S. Cl. CPC C07K 7/08; A61K 45/06; A61K 47/6455; C07K 19/00

(57) ABSTRACT

The present invention relates to peptides, in particular cell-penetrating peptides, and to conjugates of such cell-penetrating peptides with a therapeutic molecule, wherein the peptides comprise at least two cationic domains each comprising a plurality of lysine residues and wherein the peptides do not contain arginine residues. The present invention further relates to use of such peptides or conjugates in methods of treatment or as a medicament, especially in the treatment of genetic disorders and in particular neuromuscular diseases.

### TECHNICAL FIELD

The present invention relates to peptides, in particular cell-penetrating peptides, and to conjugates of such cell-penetrating peptides with a therapeutic molecule. The present invention further relates to use of such peptides or conjugates in methods of treatment or as a medicament, especially in the treatment of genetic disorders and in particular neuromuscular diseases.

### BACKGROUND

Nucleic acid drugs are genomic medicines with the potential to transform human healthcare. Research has indicated that such therapeutics could have applications across a broad range of disease areas including neuromuscular disease. The application of antisense oligonucleotide-based methods to modulate pre-mRNA splicing in the neuromuscular disease Duchenne muscular dystrophy (DMD), for example, has placed these monogenic disorder at the forefront of advances in precision medicine.

Whilst the field of antisense therapeutics for treating diseases resulting from splicing errors has progressed, the problem of delivering such therapeutics to the tissues which require them has hampered success. In September 2016 the Food and Drug Administration (FDA) granted accelerated approval for ‘eteplirsen’, a single-stranded oligonucleotide for modulating the splicing of exon 51, the region responsible for causing DMD. Yet the levels of dystrophin restoration were disappointing with only approximately 1% of normal dystrophin levels. Comparisons with the allelic disorder Becker muscular dystrophy and experiments in the mdx mouse have indicated that homogenous sarcolemmal dystrophin expression of at least ˜15% of wild-type is needed to protect muscle against exercise induced damage. The cause of the poor effectiveness is speculated to be due to poor delivery.

Therefore there is a strong and urgent need to improve the delivery of antisense oligonucleotides in order to provide a more effective therapy for devastating genetic diseases such as DMD.

The use of viruses as delivery vehicles has been suggested, however their use is limited due to the immunotoxicity of the viral coat protein and potential oncogenic effects. Alternatively, a range of non-viral delivery vectors have been developed, amongst which peptides have shown the most promise due to their small size, targeting specificity and ability of trans-capillary delivery of large bio-cargoes. Several peptides have been reported for their ability to permeate cells either alone or carrying a bio-cargo.

For several years, cell-penetrating peptides (CPPs) have been conjugated to single stranded oligonucleotides (in particular charge neutral phosphorodiamidate morpholino oligomers (PMO) and peptide nucleic acids (PNA)) in order to enhance the cell delivery of such therapeutics by effectively carrying them across cell membranes to reach their pre-mRNA target sites in the cell nucleus. It has been shown that PMO therapeutics conjugated to certain arginine-rich CPPs (known as P-PMOs or peptide-PMOs) can enhance dystrophin production in skeletal muscles following systemic administration in a mdx mouse model of DMD.

In particular, a group of CPPs were developed having two arginine-rich sequences separated by a central short hydrophobic sequence. These peptides were designed to improve serum stability whilst maintaining a relatively high level of exon skipping, initially by attachment to a PNA therapeutic. Further derivatives of these peptides were designed as conjugates with PMOs, which were shown to lead to body-wide skeletal muscle dystrophin production following systemic administration in mice. However, despite these CPPs being efficacious in delivery, their therapeutic application has been restricted by their associated toxicity.

Alternative cell-penetrating peptides having only a single arginine rich domain such as R₆Gly have also been produced. These CPPs have been used to produce peptide conjugates with reduced toxicities, but in contrast to the dual arginine-rich domain CPPs, the R₆Gly conjugates exhibited low efficacy.

Accordingly, the currently available CPPs have not yet been demonstrated as suitable for use in human treatments for diseases such as DMD. They have proven to be either ineffective or too toxic.

The challenge in the field of cell-penetrating peptide technology has been to de-couple efficacy and toxicity. Work on CPPs so far has suggested that peptides with high numbers of Arginine residues are key to cell penetration capability, with much evidence teaching towards arginine being essential. Although high numbers of arginine residues increase toxicity of CPPs, little research has been conducted on peptides in which arginine has been substituted with different amino acids. An approach to address toxicity could involve the replacement of arginine with other basic amino acids but the research that has been done on this has concluded that such alternative peptides are ineffective.

The present inventors have now identified, synthesized and tested a number of improved CPPs which address at least this problem.

### SUMMARY OF THE INVENTION

According to a first aspect of the present invention there is provided a peptide having a total length of 40 amino acid residues or less, the peptide comprising at least two cationic domains and at least one hydrophobic domain, wherein the at least two cationic domains each comprise a plurality of lysine residues and wherein the peptide does not contain arginine residues.

According to a second aspect of the present invention, there is provided a conjugate comprising a peptide according to the first aspect, covalently linked to a therapeutic molecule.

According to a third aspect there is provided a pharmaceutical composition comprising the conjugate according to the second aspect.

According to a fourth aspect there is provided a conjugate according to the second aspect, or pharmaceutical composition according to the third aspect for use as a medicament.

According to a fifth aspect there is provided a conjugate according to the second aspect, or pharmaceutical composition according to the third aspect for use in the treatment of a diseases of the neuromuscular system or musculoskeletal system, preferably genetic diseases of the neuromuscular system or musculoskeletal system, preferably hereditary genetic diseases of the neuromuscular system or musculoskeletal system.

Accordingly, it will be appreciated that delivering a conjugate or pharmaceutical composition in accordance with the present invention directly to affected tissues would be particularly advantageous. The inventors have found that the conjugate in accordance with the present invention demonstrates positive biodistribution and delivery to specific tissues such as skeletal and cardiac tissues (as shown in the Examples).

The inventors have surprisingly found that it is possible to replace arginine with lysine in a cell penetrating peptide, and still achieve good cell penetrance with reduced toxicity. In fact, the inventors have found that CPPs which contain no arginine residues and instead contain lysine residues have much reduced toxicity. It was previously believed that lysine would interact with cell-surface proteoglycans less effectively because it is less basic than arginine. Some past studies showed that replacement of arginine with lysine residues reduced the uptake of conjugates comprising CPPs, and further reduced endosomal escape of the conjugates within a cell, thereby resulting in poor activity. In some cases it was reported that a minimum number of arginine residues is required to induce endosomal escape of the CPP into the cytosol so that it can transport its therapeutic payload to the target which is often in the nucleus. The inventors have shown that in fact lysine rich CPPs act as potent delivery agents within a conjugate and have a wide therapeutic window, and that there is no requirement for arginine to be present. The results presented herein indicate that the arginine to lysine change in the CPPs broadened the difference between median activity and toxicity compared to prior CPPs.

Advantageously, the inventors have demonstrated that the conjugates in accordance with the present invention are capable of reducing the number of nuclear foci in a cell at concentrations that do not result in decreased cell viability. This is in contrast to control cells that induced significant cell mortality at similar concentrations (as described in the Examples section).

Suitably, the conjugates in accordance with the present invention may reduce nuclear foci in a cell by more than 30%, more than 35%, more than 40%, more than 45%, more than 50%, more than 55%, more than 60%, more than 65%, more than 70%, more than 75%, more than 80%, more than 85%, more than 90%, more than 95%, more than 96%, more than 97%, more than 98%, more than 99% or by 100%.

The inventors have also found that use of the conjugates in accordance with the present invention result in a significant reduction in toxicity as compared with control peptide (as demonstrated in the Examples section). It will be appreciated that a reduction in toxicity caused by conjugates for therapeutic purposes provides significant clinical advantages.

The invention includes any combination of the aspects and features described except where such a combination is clearly impermissible or expressly avoided.

The section headings used herein are for organisational purposes only and are not to be construed as limiting the subject matter described.

References to ‘X’ throughout denote any form of the artificial, synthetically produced amino acid aminohexanoic acid, preferably 6-aminohexanoic acid.

References to ‘B’ throughout denote the natural but non-genetically encoded amino acid beta-alanine.

References to ‘Ac’ throughout denote acetylation of the relevant peptide.

References to other capital letters throughout denote the relevant genetically encoded amino acid residue in accordance with the accepted alphabetic amino acid code.

### Cationic Domain

The present invention relates to short cell-penetrating peptides having a particular structure in which there are at least two lysine-rich cationic domains.

References to ‘cationic’ herein denote an amino acid or domain of amino acids having an overall positive charge at physiological pH.

Suitably, the peptide comprises up to 4 cationic domains, up to 3 cationic domains.

Suitably, the peptide comprises 2 cationic domains. Suitably the peptide comprises a first cationic domain and a second cationic domain.

As defined above, the peptide comprises two or more cationic domains each having a length of at least 4 amino acid residues.

Suitably, each cationic domain has a length of between 4 to 12 amino acid residues, suitably a length of between 4 to 9 amino acid residues.

Suitably, each cationic domain has a length of 4, 5, 6, 7, 8 or 9 amino acid residues.

Suitably, each cationic domain is of similar length, suitably each cationic domain is the same length.

Suitably, each cationic domain comprises cationic amino acids and may also contain polar and or nonpolar amino acids.

Non-polar amino acids may be selected from: alanine, beta-alanine, proline, glycine, cysteine, valine, leucine, isoleucine, methionine, tryptophan, phenylalanine, aminohexanoic acid. Suitably non-polar amino acids do not have a charge.

Polar amino acids may be selected from: serine, asparagine, hydroxyproline, histidine, threonine, tyrosine, glutamine. Suitably, the selected polar amino acids do not have a negative charge.

Cationic amino acids may be selected from: lysine or histidine. Suitably, cationic amino acids have a positive charge at physiological pH.

Suitably each cationic domain does not comprise anionic or negatively charged amino acid residues.

Suitably each cationic domain does not comprise any arginine residues.

Suitably each cationic domain comprises lysine, beta-alanine, and/or aminohexanoic acid residues.

Suitably each cationic domain consists of lysine, beta-alanine, and/or aminohexanoic acid residues.

Suitably, each cationic domain comprises at least 40%, at least 45%, at least 50% cationic amino acids.

Suitably, each cationic domain comprises a majority of cationic amino acids. Suitably, each cationic domain comprises at least 40%, at least 50% at least 55%, at least 60%, at least 65% at least 70%, at least 75%, at least 80%, at least 85%, at least 90% cationic amino acids.

Suitably, each cationic domain comprises an isoelectric point (pl) of at least 7.5, at least 8.0, at least 8.5, at least 9.0, at least 9.5, at least 10.0, at least 10.5, at least 11.0, at least 11.5, at least 12.0.

Suitably, each cationic domain comprises an isoelectric point (pl) of at least 10.0.

Suitably, each cationic domain comprises an isoelectric point (pl) of between 10.0 and 13.0.

In one embodiment, each cationic domain comprises an isoelectric point (pl) of between 10.4 and 12.5.

Suitably the isoelectric point of a cationic domain is calculated at physiological pH by any suitable means available in the art. Suitably, by using the IPC (www.isoelectric.org) a webbased algorithm developed by Lukasz Kozlowski, Biol Direct. 2016; 11:55. DOI: 10.1186/s13062-016-0159-9.

Suitably, each cationic domain comprises at least 1 cationic amino acid, suitably between 1-10 cationic amino acids. Suitably, each cationic domain comprises at least 2 cationic amino acids, suitably between 2-10 cationic amino acids, suitably between 2-6 cationic amino acids.

Suitably the at least 1 cationic amino acid consists of lysine. Suitably the at least 1 cationic amino acid comprised in each of the cationic domains consists of lysine. Suitably therefore all of the cationic amino acids in a given cationic domain are lysine.

Suitably therefore each cationic domain may be termed ‘lysine-rich’, any occurrence of a cationic domain herein may be replaced by a lysine rich domain. By ‘lysine rich’ it is meant that at least 40% of the cationic domain is formed of said residue. Suitably each cationic domain comprises a majority of lysine residues. Suitably, each cationic domain comprises at least 40%, at least 45%, at least 50%, at least 55%, at least 60%, at least 60%, at least 65%, least 70%, at least 75%, at least 80%, at least 85%, at least 90% lysine residues. Suitably each cationic domain comprises at least 60%, at least 70%, at least 80%, at least 90% lysine residues.

Suitably, each cationic domain comprises at least 1 lysine residue, suitably between 1-10 lysine residues. Suitably, each cationic domain comprises at least 2 lysine residues, suitably between 2-10 lysine residues, suitably between 2-6 lysine residues.

Suitably each cationic domain comprises no more than 3 contiguous lysine residues, suitably no more than 2 contiguous lysine residues.

Suitably each cationic domain may also comprise one or more beta alanine, and/or aminohexanoic acid residues.

Suitably each cationic domain may comprise between 1-2 beta alanine residues. Suitably each cationic domain may comprise between 1-2 aminohexanoic acid residues.

Suitably the peptide comprises at least two lysine rich domains. Suitably at least two lysine rich cationic domains. Suitably the peptide comprises two lysine rich domains. Suitably two lysine rich cationic domains.

In one embodiment, the peptide comprises a first cationic domain comprising lysine, beta alanine, and aminohexanoic acid residues and a second cationic domain comprising lysine, beta alanine, and aminohexanoic acid residues.

In one embodiment, the peptide comprises a first cationic domain consisting of lysine, beta alanine, and aminohexanoic acid residues and a second cationic domain consisting of lysine, beta alanine, and aminohexanoic acid residues.

In one embodiment, the peptide comprises a first lysine rich domain comprising lysine, beta alanine, and aminohexanoic acid residues and a second lysine rich domain comprising lysine, beta alanine, and aminohexanoic acid residues.

In one embodiment, the peptide comprises a first lysine rich domain consisting of lysine, beta alanine, and aminohexanoic acid residues and a second lysine rich domain consisting of lysine, beta alanine, and aminohexanoic acid residues.

Suitably, the peptide comprises at least two cationic domains, suitably these cationic domains form the arms of the peptide. Suitably, the cationic domains are located at the N and C terminus of the peptide. Suitably therefore, the cationic domains may be known as the cationic arm domains.

In one embodiment, the peptide comprises two cationic domains, wherein one is located at the N-terminus of the peptide and one is located at the C-terminus of the peptide. Suitably at either end of the peptide. Suitably no further amino acids or domains are present at the N-terminus and C-terminus of the peptide, with the exception of other groups such as a terminal modification, linker and/or therapeutic molecule. For the avoidance of doubt, such other groups may be present in addition to ‘the peptide’ described and claimed herein. Suitably therefore each cationic domain forms the terminus of the peptide. Suitably, this does not preclude the presence of a further linker group as described herein.

Suitably, the peptide may comprise up to 4 cationic domains. Suitably, the peptide comprises two cationic domains. Suitably the peptide comprises a first cationic domain and a second cationic domain.

In one embodiment, the peptide comprises two cationic domains that are both lysine rich. Suitably the peptide comprises a first lysine rich domain and a second lysine rich domain.

Suitably, the cationic domains comprise amino acid units selected from the following: B, BB, BBB, X, XX, XXX, K, KK, KKK, BK, KB, BX, XB, XK, KX, BKB, BXB, KBK, XBX, KXK, XKX, BBK, BBX, XXB, XXK, KKB, KKX, KBB, XBB, KXX, BKK, XKK, or any combination thereof.

Suitably, each cationic domain comprises any of the following sequences: KXKKBKK (SEQ ID NO. 1), KXKKBKKXK (SEQ ID NO. 2), KBKKBKK (SEQ ID NO. 3), KBKKBK (SEQ ID NO. 4), KBKK (SEQ ID NO. 5), KXKBKXK (SEQ ID NO. 6), KBKXK (SEQ ID NO. 7), KBKBK (SEQ ID NO. 8), and BKBK (SEQ ID NO. 9).

Suitably, each cationic domain comprises one of the following sequences: KXKKBKK (SEQ ID NO. 1), KXKKBKKXK (SEQ ID NO. 2), KBKKBKK (SEQ ID NO. 3), KBKKBK (SEQ ID NO. 4), KBKK (SEQ ID NO. 5), KXKBKXK (SEQ ID NO. 6), KBKXK (SEQ ID NO. 7), KBKBK (SEQ ID NO. 8), and BKBK (SEQ ID NO. 9).

Suitably, each cationic domain consists of any of the following sequences: KXKKBKK (SEQ ID NO. 1), KXKKBKKXK (SEQ ID NO. 2), KBKKBKK (SEQ ID NO. 3), KBKKBK (SEQ ID NO. 4), KBKK (SEQ ID NO. 5), KXKBKXK (SEQ ID NO. 6), KBKXK (SEQ ID NO. 7), KBKBK (SEQ ID NO. 8), and BKBK (SEQ ID NO. 9).

Suitably, each cationic domain consists of one of the following sequences: KXKKBKK (SEQ ID NO. 1), KXKKBKKXK (SEQ ID NO. 2), KBKKBKK (SEQ ID NO. 3), KBKKBK (SEQ ID NO. 4), KBKK (SEQ ID NO. 5), KXKBKXK (SEQ ID NO. 6), KBKXK (SEQ ID NO. 7), KBKBK (SEQ ID NO. 8), and BKBK (SEQ ID NO. 9).

Suitably the first cationic domain comprises any of the following sequences: KXKKBKK (SEQ ID NO. 1), KXKKBKKXK (SEQ ID NO. 2), KBKKBKK (SEQ ID NO. 3), KBKKBK (SEQ ID NO. 4), and KBKK (SEQ ID NO. 5). Suitably the first cationic domain consists of any of the following sequences: KXKKBKK (SEQ ID NO. 1), KXKKBKKXK (SEQ ID NO. 2), KBKKBKK (SEQ ID NO. 3), KBKKBK (SEQ ID NO. 4), and KBKK (SEQ ID NO. 5).

Suitably the first cationic domain comprises one of the following sequences: KXKKBKK (SEQ ID NO. 1), KXKKBKKXK (SEQ ID NO. 2), KBKKBKK (SEQ ID NO. 3), KBKKBK (SEQ ID NO. 4), and KBKK (SEQ ID NO. 5). Suitably the first cationic domain consists of any of the following sequences: KXKKBKK (SEQ ID NO. 1), KXKKBKKXK (SEQ ID NO. 2), KBKKBKK (SEQ ID NO. 3), KBKKBK (SEQ ID NO. 4), and KBKK (SEQ ID NO. 5).

Suitably the second cationic domain comprises any of the following sequences: KXKBKXK (SEQ ID NO. 6), KBKXK (SEQ ID NO. 7), KBKBK (SEQ ID NO. 8), and BKBK (SEQ ID NO. 9). Suitably the second cationic domain consists of any of the following sequences: KXKBKXK (SEQ ID NO. 6), KBKXK (SEQ ID NO. 7), KBKBK (SEQ ID NO. 8), and BKBK (SEQ ID NO. 9).

Suitably the second cationic domain consists of one the following sequences: KXKBKXK (SEQ ID NO. 6), KBKXK (SEQ ID NO. 7), KBKBK (SEQ ID NO. 8), and BKBK (SEQ ID NO. 9). Suitably the second cationic domain consists of any of the following sequences: KXKBKXK (SEQ ID NO. 6), KBKXK (SEQ ID NO. 7), KBKBK (SEQ ID NO. 8), and BKBK (SEQ ID NO. 9).

Suitably each cationic domain in the peptide may be identical or different. Suitably each cationic domain in the peptide is different.

### Hydrophobic Domain

The present invention relates to short cell-penetrating peptides having a particular structure in which there is at least one hydrophobic domain.

References to ‘hydrophobic’ herein denote an amino acid or domain of amino acids having the ability to repel water or which do not mix with water.

Suitably the peptide comprises up to 3 hydrophobic domains, up to 2 hydrophobic domains. Suitably the peptide comprises 1 hydrophobic domain.

As defined above, the peptide comprises one or more hydrophobic domains each having a length of at least 3 amino acid residues.

Suitably, each hydrophobic domain has a length of between 3-6 amino acids. Suitably, each hydrophobic domain has a length of 5 amino acids.

Suitably, each hydrophobic domain may comprise nonpolar, polar, and hydrophobic amino acid residues.

Hydrophobic amino acid residues may be selected from: alanine, valine, leucine, isoleucine, phenylalanine, tyrosine, methionine, and tryptophan.

Non-polar amino acid residues may be selected from: proline, glycine, cysteine, alanine, valine, leucine, isoleucine, tryptophan, phenylalanine, methionine.

Polar amino acid residues may be selected from: Serine, Asparagine, hydroxyproline, histidine, arginine, threonine, tyrosine, glutamine.

Suitably the hydrophobic domains do not comprise hydrophilic amino acid residues.

Suitably, each hydrophobic domain comprises a majority of hydrophobic amino acid residues. Suitably, each hydrophobic domain comprises at least 70%, at least 75%, at least 80%, at least 85%, at least 90%, at least 95%, 100% hydrophobic amino acids. Suitably, each hydrophobic domain consists of hydrophobic amino acid residues.

Suitably, each hydrophobic domain comprises a hydrophobicity measurement of at least 0.3, at least 0.4, at least 0.5, at least 0.6, at least 0.7, at least 0.8, at least 0.8, at least 1.0, at least 1.1, at least 1.2, at least 1.3 on a hydrophobicity scale.

Suitably, each hydrophobic domain comprises a hydrophobicity measurement of at least 0.3, at least 0.35, at least 0.4, at least 0.45 on a hydrophobicity scale.

Suitably, each hydrophobic domain comprises a hydrophobicity measurement of at least 1.2, at least 1.25, at least 1.3, at least 1.35 on a hydrophobicity scale.

Suitably, each hydrophobic domain comprises a hydrophobicity measurement of between 0.4 and 1.4 on a hydrophobicity scale.

In one embodiment, each hydrophobic domain comprises of a hydrophobicity measurement of between 0.45 and 0.48 on a hydrophobicity scale.

In one embodiment, each hydrophobic domain comprises a hydrophobicity measurement of between 1.27 and 1.39 on a hydrophobicity scale.

Suitably, hydrophobicity is as measured by White and Wimley: W. C. Wimley and S. H. White, “Experimentally determined hydrophobicity scale for proteins at membrane interfaces” Nature Struct Biol 3:842 (1996).

Suitably, each hydrophobic domain comprises at least 3, at least 4 hydrophobic amino acid residues.

Suitably, each hydrophobic domain comprises phenylalanine, leucine, Isoleucine, tyrosine, tryptophan, arginine, proline, and glutamine residues. Suitably, each hydrophobic domain consists of phenylalanine, leucine, isoleucine, tyrosine, tryptophan, arginine, proline, and/or glutamine residues.

In one embodiment, each hydrophobic domain consists of phenylalanine, leucine, isoleucine, tyrosine, arginine and/or glutamine residues.

Suitably, the peptide comprises one hydrophobic domain. Suitably the or each hydrophobic domain is located in the centre of the peptide. Suitably, therefore, the hydrophobic domain may be known as a core hydrophobic domain. Suitably, the or each hydrophobic core domain is flanked on either side by an arm domain. Suitably the arm domains may comprise one or more cationic domains and one or more further hydrophobic domains. Suitably, each arm domain comprises a cationic domain.

In one embodiment, the peptide comprises two arm domains flanking a hydrophobic core domain, wherein each arm domain comprises a cationic domain.

In one embodiment, the peptide consists of two cationic arm domains flanking a hydrophobic core domain.

Suitably the or each hydrophobic domain comprises one of the following sequences: YQFLI

(SEQ ID NO. 10), ILFQY (SEQ ID NO. 11), YRLFI (SEQ ID NO. 12), and FQILY (SEQ ID NO. 13). Suitably the or each hydrophobic domain consists of one of the following sequences: YQFLI (SEQ ID NO. 10), ILFQY (SEQ ID NO. 11), YRLFI (SEQ ID NO. 12), and FQILY (SEQ ID NO. 13). Suitably the or each hydrophobic domain comprises one of the following sequences: YQFLI (SEQ ID NO. 10), or FQILY (SEQ ID NO. 11). Suitably the or each hydrophobic domain consists of one of the following sequences: YQFLI (SEQ ID NO. 10), or FQILY (SEQ ID NO. 13).

Suitably each hydrophobic domain in the peptide may have the same sequence or a different sequence

### Peptide

The present invention relates to short cell-penetrating peptides for use in transporting therapeutic cargo molecules in the treatment of medical conditions.

The peptide has a sequence that is a contiguous single molecule, therefore the domains of the peptide are contiguous. Suitably, the peptide comprises several domains in a linear arrangement between the N-terminus and the C-terminus. Suitably, the domains are selected from cationic domains and hydrophobic domains described above. Suitably, the peptide consists of cationic domains and hydrophobic domains wherein the domains are as defined above.

Each domain has common sequence characteristics as described in the relevant sections above, but the exact sequence of each domain is capable of variation and modification. Thus a range of sequences is possible for each domain. The combination of each possible domain sequence yields a range of peptide structures, each of which form part of the present invention. Features of the peptide structures are described below.

Suitably, a hydrophobic domain separates any two cationic domains. Suitably, each hydrophobic domain is flanked by cationic domains on either side thereof.

Suitably no cationic domain is contiguous with another cationic domain.

In one embodiment, the peptide comprises one hydrophobic domain flanked by two cationic domains in the following arrangement:
[cationic domain]-[hydrophobic domain]-[cationic domain]

Therefore, suitably the hydrophobic domain may be known as the core domain and each of the cationic domains may be known as an arm domain. Suitably, the hydrophobic arm domains flank the cationic core domain on either side thereof.

In one embodiment, the peptide consists of two cationic domains and one hydrophobic domain. Suitably wherein the peptide consists of the following structure:
[first cationic domain]-[hydrophobic domain]-[second cationic domain]

In one embodiment, the peptide consists of one hydrophobic core domain flanked by two cationic arm domains.

In one embodiment, the peptide comprises or consists of one hydrophobic domain comprising a sequence selected from: YQFLI (SEQ ID NO. 10), ILFQY (SEQ ID NO. 11), YRLFI (SEQ ID NO. 12), and FQILY (SEQ ID NO. 13), flanked by two cationic domains each comprising a sequence selected from: KXKKBKK (SEQ ID NO. 1), KXKKBKKXK (SEQ ID NO. 2), KBKKBKK (SEQ ID NO. 3), KBKKBK (SEQ ID NO. 4), KBKK (SEQ ID NO. 5), KXKBKXK (SEQ ID NO. 6), KBKXK (SEQ ID NO. 7), KBKBK (SEQ ID NO. 8), and BKBK (SEQ ID NO. 9).

In one embodiment, the peptide comprises or consists of one hydrophobic domain comprising a sequence selected from: YQFLI (SEQ ID NO. 10), and FQILY (SEQ ID NO. 13), flanked by two cationic domains each comprising a sequence selected from: KXKKBKK (SEQ ID NO. 1), KXKKBKKXK (SEQ ID NO. 2), KBKKBKK (SEQ ID NO. 3), KBKKBK (SEQ ID NO. 4), KBKK (SEQ ID NO. 5), KXKBKXK (SEQ ID NO. 6), KBKXK (SEQ ID NO. 7), KBKBK (SEQ ID NO. 8), and BKBK (SEQ ID NO. 9).

In one embodiment, the peptide comprises or consists of one hydrophobic domain comprising a sequence selected from: YQFLI (SEQ ID NO. 10), ILFQY (SEQ ID NO. 11), YRLFI (SEQ ID NO. 12), and FQILY (SEQ ID NO. 13), flanked by a first cationic domain comprising a sequence selected from: KXKKBKK (SEQ ID NO. 1), KXKKBKKXK (SEQ ID NO. 2), KBKKBKK (SEQ ID NO. 3), KBKKBK (SEQ ID NO. 4), and KBKK (SEQ ID NO. 5), and a second cationic domain comprising a sequence selected from: KXKBKXK (SEQ ID NO. 6), KBKXK (SEQ ID NO. 7), KBKBK (SEQ ID NO. 8), and BKBK (SEQ ID NO. 9).

In one embodiment, the peptide comprises or consists of one hydrophobic domain comprising a sequence selected from: YQFLI (SEQ ID NO. 10), and FQILY (SEQ ID NO. 13), flanked by a first cationic domain comprising a sequence selected from: KXKKBKK (SEQ ID NO. 1), KXKKBKKXK (SEQ ID NO. 2), KBKKBKK (SEQ ID NO. 3), KBKKBK (SEQ ID NO. 4), and KBKK (SEQ ID NO. 5), and a second cationic domain comprising a sequence selected from: KXKBKXK (SEQ ID NO. 6), KBKXK (SEQ ID NO. 7), KBKBK (SEQ ID NO. 8), and BKBK (SEQ ID NO. 9).

In any such embodiment, further groups may be present such as a linker, terminal modification and/or therapeutic molecule.

Suitably, the peptide is N-terminally modified.

Suitably the peptide is N-acetylated, N-methylated, N-trifluoroacetylated, N-trifluoromethylsulfonylated, or N-methylsulfonylated. Suitably, the peptide is N-acetylated.

Optionally, the N-terminus of the peptide may be unmodified.

In one embodiment, the peptide is N-acetylated.

Suitably, the peptide is C-terminal modified.

Suitably, the peptide comprises a C-terminal modification selected from: Carboxy-, Thioacid-, Aminooxy-, Hydrazino-, thioester-, azide, strained alkyne, strained alkene, aldehyde-, thiol or haloacetyl-group.

Advantageously, the C-terminal modification provides a means for linkage of the peptide to the therapeutic molecule.

Accordingly, the C-terminal modification may comprise the linker and vice versa. Suitably, the C-terminal modification may consist of the linker or vice versa. Suitable linkers are described herein elsewhere.

Suitably, the peptide comprises a C-terminal carboxyl group.

Suitably, the C-terminal carboxyl group is provided by a cysteine, glycine or beta-alanine residue.

In one embodiment, the C terminal carboxyl group is provided by a cysteine residue.

Suitably, the C terminal cysteine residue is a linker.

Suitably, therefore each cationic domain may further comprise an N or C terminal modification. Suitably the cationic domain at the C terminus comprises a C-terminal modification. Suitably the cationic domain at the N terminus comprises a N-terminal modification. Suitably, the cationic domain at the C terminus comprises a linker group, suitably, the cationic domain at the C terminus comprises a C-terminal beta-alanine. Suitably, the cationic domain at the N terminus is N-acetylated.

The peptide of the present invention is defined as having a total length of 40 amino acid residues or less. The peptide may therefore be regarded as an oligopeptide.

Suitably, the peptide has a total length of between 3-35 amino acid residues, suitably of between 5-30 amino acid residues in length, of between 10-25 amino acid residues in length, of between 13-23 amino acid residues in length, of between 15-20 amino acid residues length.

Suitably, the peptide has a total length of at least 12, at least 13, at least 14, at least 15 amino acid residues.

Suitably the peptide is less than 35 amino acid residues in length, less than 30 amino acid residues in length, less than 25 amino acid residues in length, less than 20 amino acid residues in length.

Suitably the peptide is capable of penetrating cells. The peptide may therefore be regarded as a cell-penetrating peptide.

Suitably, the peptide is for attachment to a therapeutic molecule. Suitably, the peptide is for transporting a therapeutic molecule into a target cell. Suitably, the peptide is for delivering a therapeutic molecule into a target cell. The peptide may therefore be regarded as a carrier peptide.

Suitably, the peptide is capable of penetrating into cells and tissues, suitably into the nucleus of cells. Suitably into muscle tissues.

Suitably, the peptide may be selected from any of the following sequences:

(SEQ ID NO. 14)
KXKKBKK FQILY KBKXK (ERA 5.2)
(SEQ ID NO. 15)
KXKKBKKXK YQFLI KXKBKXK (ERA 5.1)
(SEQ ID NO. 16)
KBKKBKK FQILY KBKXK
(SEQ ID NO. 17)
KBKKBKK FQILY KBKBK (ERA 5.3)
(SEQ ID NO. 18)
KBKK YQFLI KBKXK (ERA 5.4)
(SEQ ID NO. 19)
KBKKBK FQILY BKBK (ERA 5.5)

In one embodiment, the peptide consists of the following sequence: KXKKBKK FQILY KBKXK (SEQ ID NO. 14).

In one embodiment, the peptide consists of the following sequence: KXKKBKKXK YQFLI KXKBKXK (SEQ ID NO. 15).

### Conjugate

The peptide of the invention may be covalently linked to a therapeutic molecule in order to provide a conjugate.

The therapeutic molecule may be any molecule for treatment of a disease. The therapeutic molecule may be selected from: a nucleic acid, peptide nucleic acid, antisense oligonucleotide (such as PNA, PMO), mRNA, gRNA (for example in the use of CRISPR/Cas9 technology), short interfering RNA, micro RNA, antagomiRNA, peptide, cyclic peptide, protein, pharmaceutical, drug, or nanoparticle.

In one embodiment, the therapeutic molecule is an antisense oligonucleotide.

Suitably the antisense oligonucleotide is comprised of a phosphorodiamidate morpholino oligonucleotide (PMO).

Alternatively the oligonucleotide may be a modified PMO or any other charge-neutral oligonucleotide such as a peptide nucleic acid (PNA), a chemically modified PNA such as a gamma-PNA (Bahal, Nat. Comm. 2016), oligonucleotide phosphoramidate (where the nonbridging oxygen of the phosphate is substituted by an amine or alkylamine such as those described in WO2016028187A1, or any other partially or fully charge-neutralized oligonucleotide.

The therapeutic antisense oligonucleotide sequence may be selected from any that are available, for example antisense oligonucleotides for exon skipping in DMD are described in https://research-repository.uwa.edu.au/en/publications/antisense-oligonucleotide-inducedexon-skipping-across-the-human-, or a therapeutic antisense oligonucleotide complementary to the ISSN1 or IN7 sequence for the treatment of SMA are described in Zhou, H G T, 2013; and Hammond et al, 2016; and Osman et al, HMG, 2014.

Optionally, lysine residues may be added to one or both ends of a therapeutic molecule (such as a PMO or PNA) before attachment to the peptide to improve water solubility.

Suitably the therapeutic molecule has a molecular weight of less than 15,000 Da, less than 10,000 Da, less than 9,000 Da, less than 8,000 Da, less than 7,000 Da, less than 6,000 Da, less than 5,000 Da, less than 5,000 Da, less than 4,000 Da, less than 3,000 Da, less than 2,000 Da or suitably less than 1,000 Da.

Suitably, the peptide is covalently linked to the therapeutic molecule at the C-terminus.

Suitably, the peptide is covalently linked to the therapeutic molecule through a linker if required. The linker may act as a spacer to separate the peptide sequence from the therapeutic molecule.

The linker may be selected from any suitable sequence.

Suitably the linker is present between the peptide and the therapeutic molecule. Suitably the linker is a separate group to the peptide and the therapeutic molecule. Accordingly, the linker may comprise artificial amino acids.

In one embodiment, the conjugate comprises the peptide covalently linked via a linker to a therapeutic molecule. In one embodiment, the conjugate comprises the following structure:
[peptide]-[linker]-[therapeutic molecule]

In one embodiment, the conjugate consists of the following structure:
[peptide]-[linker]-[therapeutic molecule]

Suitably any of the peptides listed herein may be used in a conjugate according to the invention.

Suitable linkers include, for example, a C-terminal cysteine residue that permits formation of a disulphide, thioether or thiol-maleimide linkage, a C-terminal aldehyde to form an oxime, a click reaction or formation of a morpholino linkage with a basic amino acid on the peptide or a carboxylic acid moiety on the peptide covalently conjugated to an amino group to form a carboxamide linkage.

Suitably, the linker is between 1-5 amino acids in length. Suitably the linker may comprise any linker that is known in the art.

Suitably the linker is selected from any of the following sequences: G, BC, XC, C, GGC, BBC, BXC, XBC, X, XX, B, BB, BX and XB.

In one embodiment, the linker is cysteine.

Suitably, in embodiments where the peptide further comprises a linker, suitably any of the above peptide sequences further comprise a linker at the C-terminus. Suitably any of the above peptide sequences may comprise cysteine linker at the C-terminus. Suitably in such an embodiment, the peptide may be selected from any of the following sequences:

(SEQ ID NO. 20)
KXKKBKK FQILY KBKXK-C
(SEQ ID NO. 21)
KXKKBKKXK YQFLI KXKBKXK-C
(SEQ ID NO. 22)
KBKKBKK FQILY KBKXK-C
(SEQ ID NO. 23)
KBKKBKK FQILY KBKBK-C
(SEQ ID NO. 24)
KBKK YQFLI KBKXK-C
(SEQ ID NO. 25)
KBKKBK FQILY BKBK-C

In one embodiment, the peptide is conjugated to the therapeutic molecule through a disulphide, thioether or thiol-maleimide linkage.

The linker of the conjugate may form part of the therapeutic molecule to which the peptide is attached. Alternatively, the attachment of the therapeutic molecule may be directly linked to the C-terminus of the peptide. Suitably, in such embodiments, no linker is required.

Alternatively, the peptide may be chemically conjugated to the therapeutic molecule. Chemical linkage may be via a disulphide, alkenyl, alkynyl, aryl, ether, thioether, triazole, amide, carboxamide, urea, thiourea, semicarbazide, carbazide, hydrazine, oxime, phosphate, phosphoramidate, thiophosphate, boranophosphate, iminophosphates, or thiol-maleimide linkage, for example.

Optionally, cysteine may be added at the N-terminus of a therapeutic molecule to allow for disulphide bond formation to the peptide, or the N-terminus may undergo bromoacetylation for thioether conjugation to the peptide.

The peptide of the invention may equally be covalently linked to an imaging molecule in order to provide a conjugate.

Suitably, the imaging molecule may be any molecule that enables visualisation of the conjugate. Suitably, the imaging molecule may indicate the location of the conjugate. Suitably the location of the conjugate in vitro or in vivo. Suitably, there is provided a method of monitoring the location of a conjugate comprising an imaging molecule comprising: administering the conjugate to a subject and imaging the subject to locate the conjugate.

Examples of imaging molecules include detection molecules, contrast molecules, or enhancing molecules. Suitable imaging molecules may be selected from radionuclides; fluorophores; nanoparticles (such as a nanoshell); nanocages; chromogenic agents (for example an enzyme), radioisotopes, dyes, radiopaque materials, fluorescent compounds, and combinations thereof.

Suitably imaging molecules are visualised using imaging techniques, these may be cellular imaging techniques or medical imaging techniques. Suitable cellular imaging techniques include image cytometry, fluorescent microscopy, phase contrast microscopy, SEM, TEM, for example. Suitable medical imaging techniques include X-ray, fluoroscopy, MRI, scintigraphy, SPECT, PET, CT, CAT, FNRI, for example.

In some cases, the imaging molecule may be regarded as a diagnostic molecule. Suitably, a diagnostic molecule enables the diagnosis of a disease using the conjugate. Suitably, diagnosis of a disease may be achieved through determining the location of the conjugate using an imaging molecule. Suitably, there is provided a method of diagnosis of a disease comprising administering an effective amount of a conjugate comprising an imaging molecule to a subject and monitoring the location of the conjugate.

Suitably, further details such as the linkage of a conjugate comprising an imaging molecule are the same as those described above in relation to a conjugate comprising a therapeutic molecule.

Suitably, the peptide of the invention may be covalently linked to a therapeutic molecule and an imaging molecule in order to provide a conjugate.

Suitably the conjugate is capable of penetrating into cells and tissues, suitably into the nucleus of cells. Suitably into muscle tissues.

### Pharmaceutical Composition

The conjugate of the invention may formulated into a pharmaceutical composition.

Suitably the pharmaceutical composition comprises a conjugate of the invention.

Suitably, the pharmaceutical composition may further comprise a pharmaceutically acceptable diluent, adjuvant or carrier.

Suitable pharmaceutically acceptable diluents, adjuvants and carriers are well known in the art.

As used herein, the phrase “pharmaceutically acceptable” refers to those ligands, materials, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier”, as used herein, refers to a pharmaceutically acceptable material, formulation or vehicle, such as a liquid or solid filler, diluent, excipient, solvent or encapsulating material, involved in carrying or transporting the conjugate from one organ or portion of the body, to another organ or portion of the body. Each cell-penetrating peptide must be “acceptable” in the sense of being compatible with the other components of the composition e.g. the peptide and therapeutic molecule, and not injurious to the individual. Lyophilized compositions, which may be reconstituted and administered, are also within the scope of the present composition.

Pharmaceutically acceptable carriers may be, for example, excipients, vehicles, diluents, and combinations thereof. For example, where the compositions are to be administered orally, they may be formulated as tablets, capsules, granules, powders, or syrups; or for parenteral administration, they may be formulated as injections, drop infusion preparations, or suppositories. These compositions can be prepared by conventional means, and, if desired, the active compound (i.e. conjugate) may be mixed with any conventional additive, such as an excipient, a binder, a disintegrating agent, a lubricant, a corrigent, a solubilizing agent, a suspension aid, an emulsifying agent, a coating agent, or combinations thereof.

It should be understood that the pharmaceutical compositions of the present disclosure can further include additional known therapeutic agents, drugs, modifications of compounds into prodrugs, and the like for alleviating, mediating, preventing, and treating the diseases, disorders, and conditions described herein under medical use.

Suitably, the pharmaceutical composition is for use as a medicament. Suitably for use as a medicament in the same manner as described herein for the conjugate. All features described herein in relation to medical treatment using the conjugate apply to the pharmaceutical composition.

Accordingly, in a further aspect of the invention there is provided a pharmaceutical composition according to the fourth aspect for use as a medicament. In a further aspect, there is provided a method of treating a subject for a disease condition comprising administering an effective amount of a pharmaceutical composition according to the fourth aspect to the subject.

### Medical Uses

The conjugate comprising the peptide of the invention may be used as a medicament for the treatment of a disease.

The medicament may be in the form of a pharmaceutical composition as defined above.

A method of treatment of a patient or subject in need of treatment for a disease condition is also provided, the method comprising the step of administering a therapeutically effective amount of the conjugate to the patient or subject.

Suitably, the medical treatment requires delivery of the therapeutic molecule into a cell, suitably into the nucleus of the cell.

Diseases to be treated may include any disease where improved penetration of the cell and/or nuclear membrane by a therapeutic molecule may lead to an improved therapeutic effect.

Suitably, the conjugate is for use in the treatment of diseases of the neuromuscular system.

Conjugates comprising peptides of the invention are suitable for the treatment of genetic diseases of the neuromuscular system. Conjugates comprising peptides of the invention are suitable for the treatment of genetic neuromuscular diseases. In a suitable embodiment, there is provided a conjugate according to the second aspect for use in the treatment of genetic diseases of the neuromuscular system. Suitably, the conjugate is for use in the treatment of hereditary genetic diseases. Suitably, the conjugate is for use in the treatment of hereditary genetic diseases of the neuromuscular system. Suitably, the conjugate is for use in the treatment of hereditary genetic neuromuscular diseases. Suitably, the conjugate is for use in the treatment of hereditary X-linked genetic diseases of the neuromuscular system. Suitably, the conjugate is for use in the treatment of hereditary X-linked neuromuscular diseases.

Suitably the conjugate is for use in the treatment of a disease selected from: Duchenne Muscular Dystrophy (DMD), Bucher Muscular Dystrophy (BMD), Menkes disease, Beta-thalassemia, dementia, Parkinson's Disease, Spinal Muscular Atrophy (SMA), myotonic dystrophy (DM1 or DM2), Huntington's Disease, Hutchinson-Gilford Progeria Syndrome, Ataxia-telangiectasia, or cancer.

Suitably, the conjugate is for use in the treatment of diseases caused by splicing deficiencies. In such embodiments, the therapeutic molecule may comprise an oligonucleotide capable of preventing or correcting the splicing defect and/or increasing the production of correctly spliced mRNA molecules.

Suitably, the conjugate in accordance with the present invention is capable of inducing splicing corrections in mRNA molecules by up to 10%, up to 20%, up to 30%, up to 40%, up to 50%, up to 60%, up to 70%, up to 80%, up to 90% or up to 100%.

Suitably, the conjugate in accordance with the present invention is capable of inducing splicing corrections in mRNA molecules by between 30 and 90% at a doses that result in less toxicity as compared to a control peptide (as described in the Examples section).

Suitably, the patient or subject to be treated may be any animal or human. Suitably, the patient or subject may be a non-human mammal. Suitably the patient or subject may be male or female. In one embodiment, the subject is male.

Suitably, the patient or subject to be treated may be any age. Suitably the patient or subject to be treated is aged between 0-40 years, suitably 0-30, suitably 0-25, suitably 0-20 years of age.

Suitably, the conjugate is for administration to a subject systemically for example by intramedullary, intrathecal, intraventricular, intravitreal, enteral, parenteral, intravenous, intraarterial, intramuscular, intratumoral, subcutaneous oral or nasal routes.

In one embodiment, the conjugate is for administration to a subject intravenously.

In one embodiment, the conjugate is for administration to a subject intravenously by injection.

Suitably, the conjugate is for administration to a subject in a “therapeutically effective amount”, by which it is meant that the amount is sufficient to show benefit to the individual. The actual amount administered, and rate and time-course of administration, will depend on the nature and severity of the disease being treated. Decisions on dosage are within the responsibility of general practitioners and other medical doctors. Examples of the techniques and protocols can be found in Remington's Pharmaceutical Sciences, 20th Edition, 2000, pub. Lippincott, Williams & Wilkins.

Exemplary doses may be between 0.01 mg/kg and 50 mg/kg, 0.05 mg/kg and 40 mg/kg, 0.1 mg/kg and 30 mg/kg, 0.5 mg/kg and 18 mg/kg, 1 mg/kg and 16 mg/kg, 2 mg/kg and 15 mg/kg, 5 mg/kg and 10 mg/kg, 10 mg/kg and 20 mg/kg, 12 mg/kg and 18 mg/kg, 13 mg/kg and 17 mg/kg.

Advantageously, the dosage of the conjugates of the present invention is an order of magnitude lower than the dosage required to see any effect from the therapeutic molecule alone.

Suitably, after administration of the conjugates of the present invention, one or more markers of toxicity are significantly reduced compared to prior conjugates using currently available peptide carriers.

Suitable markers of toxicity may be markers of nephrotoxicity.

Suitable markers of toxicity include KIM-1, NGAL, BUN, creatinine, alkaline phosphatase, alanine transferase, and aspartate aminotransferase.

Suitably the level of at least one of KIM-1, NGAL, and BUN is reduced after administration of the conjugates of the present invention when compared to prior conjugates using currently available peptide carriers.

Suitably the levels of each of KIM-1, NGAL, and BUN are reduced after administration of the conjugates of the present invention when compared to prior conjugates using currently available peptide carriers.

Suitably, the levels of the or each marker/s is significantly reduced when compared to prior conjugates using currently available peptide carriers.

Suitably the levels of the or each marker/s is reduced by up to 5%, 10%, 15%, 20%, 25%, 30%, 35%, 40%, 45%, 50% after administration of the conjugates of the present invention when compared to prior conjugates using currently available peptide carriers.

Advantageously, the toxicity of the peptides and therefore the resulting conjugates is significantly reduced compared to prior cell-penetrating peptides and conjugates. In particular, KIM-1 and NGAL-1 are markers of toxicity and these are significantly reduced by up to 120 times compared to prior conjugates using currently available peptide carriers.

### Nucleic Acids and Hosts

Peptides of the invention may be produced by any standard protein synthesis method, for example chemical synthesis, semi-chemical synthesis or through the use of expression systems.

Accordingly, the present invention also relates to the nucleotide sequences comprising or consisting of the DNA coding for the peptides, expression systems e.g. vectors comprising said sequences accompanied by the necessary sequences for expression and control of expression, and host cells and host organisms transformed by said expression systems.

Accordingly, a nucleic acid encoding a peptide according to the present invention is also provided.

Suitably, the nucleic acids may be provided in isolated or purified form.

An expression vector comprising a nucleic acid encoding a peptide according to the present invention is also provided.

Suitably, the vector is a plasmid.

Suitably the vector comprises a regulatory sequence, e.g. promoter, operably linked to a nucleic acid encoding a peptide according to the present invention. Suitably, the expression vector is capable of expressing the peptide when transfected into a suitable cell, e.g. mammalian, bacterial or fungal cell.

A host cell comprising the expression vector of the invention is also provided.

Expression vectors may be selected depending on the host cell into which the nucleic acids of the invention may be inserted. Such transformation of the host cell involves conventional techniques such as those taught in Sambrook et al [Sambrook, J., Russell, D. (2001) Molecular Cloning: A Laboratory Manual, Cold Spring Harbor Laboratory Press, NY, USA]. Selection of suitable vectors is within the skills of the person knowledgeable in the field. Suitable vectors include plasmids, bacteriophages, cosmids, and viruses.

The peptides produced may be isolated and purified from the host cell by any suitable method e.g. precipitation or chromatographic separation e.g. affinity chromatography.

Suitable vectors, hosts and recombinant techniques are well known in the art.

In this specification the term “operably linked” may include the situation where a selected nucleotide sequence and regulatory nucleotide sequence are covalently linked in such a way as to place the expression of a nucleotide coding sequence under the control of the regulatory sequence, as such, the regulatory sequence is capable of effecting transcription of a nucleotide coding sequence which forms part or all of the selected nucleotide sequence. Where appropriate, the resulting transcript may then be translated into a desired peptide.

### BRIEF DESCRIPTION OF THE DRAWINGS

Certain embodiments of the present invention will now be described with reference to the following figures and tables in which:

FIG. 1: shows the comparative delivery of conjugates to target tissues in DM1 adult mice (8-12-week-old HSA-LR mice). Evaluation of ERA5.1-CAG7 and ERA5.2-CAG7 biodistribution reveals optimal delivery to critically affected tissues in DM1 two weeks after 30 mg/kg (A) and 7.5 mg/kg (B) in comparison with naked PMO and a benchmark conjugate (R6Gly-CAG7, which contains a peptide being used by Sarepta in a Clinical Trial with DMD patients) and Naked PMO (3×200 mg/kg) (all conjugates comprise the same PMOCAG7 sequence). The data shows that two weeks after 30 mg/kg the concentration of PMO in muscle tissues were still >1 nM vs the low pM detected after naked PMO injections (despite of the >20-fold difference in molarity of naked PMO vs conjugate treatments). Data are expressed as mean+/−SEM.

FIG. 2: shows CPP-PMO conjugates of the invention correct splicing defects causing DM1 pathology. Systemic delivery of conjugates at 30 mg/kg (IV, tail vein) corrected mis-splicing of Mbnl-dependent transcripts in gastrocnemius of in DM1 adult mice (8-12-week-old HSA-LR mice). RT-PCR analyses of the splicing of Mbnl1 exon 5 (A) and Clcn1 exon 7a (B) (the most widely used DM1 biomarkers) are shown (data are media±SEM).

FIG. 3: shows HSA transcript levels (containing the toxic RNA repetitive sequence) normalized to P0 2 weeks after treatment with conjugates of the invention (IV administration, 30 mg/kg) in 8-12-week HSA-LR mice in comparison with a benchmark conjugate, R6Gly-CAG7 (data are media±SEM).

FIG. 4: shows the effect on myotonia grade in HSA-LR mice (8-12-week-old) after administration (IV, tail vein) of conjugates of peptides of the invention with PMO CAG7 therapeutic compared to a benchmark conjugate (R6Gly-CAG7). ERA5.1-CAG7 and ERA5.2-CAG7 correct myotonia wild type levels 1 week after 30 mg/kg treatment (error bars, SEM).

FIG. 5: shows the Kim-1 urinary marker levels (biomarker of kidney toxicity) of conjugates of prior PIP peptides compared to conjugates of peptides of the invention with PMO therapeutics 2 days or 7 days after administration of 7.5 mg/kg or 30 mg/kg. Toxicology analysis showed no significant changes at doses that were able to normalize DM1 phenotype (according to FIGS. 3 and 4). Kim-1 was measured by ELISA (R&D cat #MKM100) with samples diluted to fit within standard curve. Values were normalised to urinary creatinine levels to account for urine protein concentration. Kim-1 levels were similar to saline control injections in comparison to the fold increases induced by the prior Pip series of peptide carriers.

FIG. 6: shows the reduction in the number of toxic DMPK foci detected by fluorescence in situ hybridization using a (CAG) 5 Cy3 labelled probe that detects mutant DMPK RNA 48 h after treatments with the conjugates of the invention in DM1 myoblasts containing 2600 repeats in the 3′UTR of DMPK mRNA (immortalized myoblasts from DM1 patients. Results are shown 48 hours after transfection at doses (10 uM is shown) of different conjugates that did not decrease cell viability of myoblasts or hepatocytes.

FIG. 7: shows cell viability after DM1 patient myoblasts with 2600 CTG repeats (A) or wild type hepatocytes (B) are 48 hours transfected with different ERA5-[CAG]7 conjugates and comparative conjugates. ERA5-CAG7 PMO conjugates concentrations can be increased several fold from therapeutic levels (according to FIG. 6) without causing cell death in hepatocytes, in contrast to conjugates formed with prior peptide carriers; Pip6a and Pip9b2. (error bars, SEM).

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The reader's attention is directed to all papers and documents which are filed concurrently with or previous to this specification in connection with this application and which are open to public inspection with this specification, and the contents of all such papers and documents are incorporated herein by reference.

### EXAMPLES

### Methods

### Peptide Synthesis

Peptide chains were elongated on a 0.1 mmol scale using a CEM Liberty Blue™ microwave Peptide Synthesizer (Buckingham, UK) and Fmoc chemistry following manufacturer's recommendations. Coupling condition utilised PyBOP and DIEA (5 and 10 equivalent respectively) while Fmoc removal used 20% piperidine in DMF. Once synthesis was complete, the resin was washed with DMF (3×5 mL) and the N-terminus of the solid phase bound peptide was acetylated with acetic anhydride in the presence of DIEA at room temperature. After acetylation of the N-terminus, the peptide resin was washed with DMF (3×5 mL) and DCM (3×5 mL). After drying the peptidyl resin, the peptide was cleaved from the solid support by treatment with a cleavage cocktail consisting of trifluoroacetic acid (TFA):H₂O:triisopropylsilane (TIS):2,3′-(ethylenedioxy)diethanethiol (94%:2.5%:2.5%:1%, 10 mL/g) for 1 h at room temperature followed by the typical diethyl ether precipitation, HPLC analysis and MALDI-TOF characterisation. Peptides were purified by 1260 Infinity II preparative HPLC Agilent system on an RP-C18 column (21.2×250 mm, Phenomenex) using a linear gradient (5 to 50 over 30 min) of 0.1% TFA CH₃CN in 0.1% TFA/H₂O with a flow rate of 15 mL/min.

### Synthesis of PMO-Peptide Conjugates Vs Maleimide Conjugation

A 21-mer PMO antisense sequence for triplet repeat sequences (CAGCAGCAGCAGCAGCAGCAG (SEQ ID NO: 26) otherwise known as CAG7 was purchased from Gene Tools (USA). Conjugation of peptides was carried out by dissolving PMO (2000 nmole) in 50 mM sodium phosophate buffer (pH 7.2) containing 20% acetonitrile at a concentration of 4 mM. A 2-fold molar excess of the cross linker 3-maleimidopropionic acid N-hydroxysuccinimide ester (GMBS, Thermo Scientific) was added and the reaction mixture was kept at room temperature for 1 hour. Maleimide functionalised PMO was then purified using PD miditrap G-25 column, lyophilised and then dissolved in 50 mM sodium phosphate buffer (pH 6.5) containing 20% acetonitrile at a 4 mM concentration. A 2 equivalent of the peptide was added to the maleimide-PMO and allowed to incubate at room temperature for 1 hours based on HPLC monitoring of the reaction. This solution was purified by Ion exchange chromatography using a converted Gilson HPLC system. The PMO-peptide conjugates were purified on an ion exchange column (prepacked Resource S, GE Healthcare) using a linear gradient of sodium phosphate buffer (25 mM, pH 7.0) containing 20% CH₃CN. A sodium chloride solution (1 M) was used to elute the conjugate from the column at a flow rate of 6 mL/min. The fractions were manually collected, and the desired compound were combined and desalted immediately. The removal of excess salts from the peptide-PMO conjugate was afforded through the filtration of the fractions collected after ion exchange using an Amicon ultra-15 3K centrifugal filter device. The conjugate was lyophilized and analyzed by MALDI-TOF and RP-HPLC. The conjugates were dissolved in sterile water and filtered through a 0.22 m cellulose acetate membrane before use. The concentration of peptide-PMO was determined by the molar absorption of the conjugates at 265 nm in 0.1 N HCl solution. Average yield was around 20% calculated from PMO starting materials.

TABLE 1
Table 1: peptides as synthesised for testing
in the examples. Conjugates formed with R6Gly,
Pip6a and Pip9b2 are comparative.
Ac indicates N terminal acetyl group,
C indicates Cysteine linker.
Gly indicates glycine linker.

| | Sequence | |
|---|---|---|
| | ID | |
| | NO. | Sequence Tested (with |
| Peptide | incorpo- | additional C and N |
| Number | rated | terminal modifications) |
| ERA5.1 | 27 | AC-KXKKBKKXK YQFLI |
| | | KXKBKXK-C |
| ERA5.2 | 41 | Ac- KXKKBKK FQILY KBKXK-C |
| ERA5.3 | 29 | Ac-KBKKBKK FQILY KBKBK-C |
| ERA5.4 | 30 | AC-KBKK YQFLI KBKXK-C |
| ERA5.5 | 31 | Ac-KBKKBK FQILY BKBK-C |
| R6Gly | 32 | Ac-RRRRRR-Gly |
| Pip6a | 33 | Ac-RXRRBRRXR-YQFLI-RXRBRXR-C |
| Pip9b2 | 34 | Ac-RXRRBRR-FQILY-RBRXR-C |

### Synthesis of a Library of Peptide-PMO Conjugates

A 21-mer PMO antisense sequence for triplet repeat sequences (CAGCAGCAGCAGCAGCAGCAG (SEQ ID NO. 26) otherwise known as [CAG]₇ was used.

The PMO sequence targeting CUG/CTG expanded repeats (5′-CAGCAGCAGCAGCAGCAGCAG-3′ (SEQ ID NO.26)) was purchased from Gene Tools LLC. This is a [CAG]₇ PMO as referenced elsewhere herein. The peptide was conjugated to the 3′-end of the PMO through its C-terminal carboxyl group. This was achieved using 2.5 and 2 equivalents of PyBOP and HOAt in NMP respectively in the presence of 2.5 equivalents of DIPEA and 2.5 fold excess of peptide over PMO dissolved in DMSO was used. In general, to a solution of peptide (2500 nmol) in N-methylpyrrolidone (NMP, 80 μL) were added PyBOP (19.2 μL of 0.3 M in NMP), HOAt in (16.7 μL of 0.3 M NMP), DIPEA (1.0 mL) and PMO (180 μL of 10 mM in DMSO). The mixture was left for 2.5 h at 40° C. This solution was purified by Ion exchange chromatography using a converted Gilson HPLC system. The PMO-peptide conjugates were purified on an ion exchange column (Resource S 4 mL, GE Healthcare) using a linear gradient of sodium phosphate buffer (25 mM, pH 7.0) containing 20% CH₃CN. A sodium chloride solution (1 M) was used to elute the conjugate from the column at a flow rate of either 4 mL min⁻¹ or 6 mL min⁻¹. The fractions containing the desired compound were combined desalted immediately. The removal of excess salts from the peptide-PMO conjugate was afforded through the filtration of the fractions collected after ion exchange using an Amicon® ultra-15 3K centrifugal filter device. The conjugate was lyophilized and analyzed by MALDI-TOF. The conjugates were dissolved in sterile water and filtered through a 0.22 μm cellulose acetate membrane before use. The concentration of peptide-PMO was determined by the molar absorption of the conjugates at 265 nm in 0.1 N HCl solution.

### Animal Model and ASO Injections

Experiments were carried out in the University of Oxford according to UK legislation. The intravenous injections in HSA-LR mice were performed by single via the tail vein. Doses of 7.5, 30 or 40 mg/kg of peptide-PMO-CAG7 or 200 mg/kg of PMO were diluted in 0.9% saline and given at a volume of 5-6 μL/g of body weight. Myotonia was evaluated and tissues were harvested 2 weeks after the last injection.

### Cell Culture and Peptide-PMO Treatment

Immortalized myoblasts from healthy individual or DM1 patient with 2600 CTG repeats were cultivated in a growth medium consisting of a mix of M199:DMEM (1:4 ratio; Life technologies) supplemented with 20% FBS (Life technologies), 50 μg/ml gentamycin (Life technologies), 25 μg/ml fetuin, 0.5 ng/ml bFGF, 5 ng/ml EGF and 0.2 μg/ml dexamethasone (Sigma-Aldrich). Myogenic differentiation was induced by switching confluent cell cultures to DMEM medium supplemented with 5 μg/ml insulin (Sigma-Aldrich) for myoblasts. For treatment, WT or DM1 cells are differentiated for 4 days. Then, medium was changed with fresh differentiation medium with peptide-PMO conjugates at a 1, 2, 5 10, 20 or 40 UM concentration. Cells were harvested for analysis 48 h after treatment.

RNA Isolation, RT-PCR and qPCR Analysis

For mice tissues: prior to RNA extraction, muscles were disrupted in TriReagent (Sigma-Aldrich) using Fastprep system and Lysing Matrix D tubes (MP biomedicals). For human cells: prior to RNA extraction, cells were lysed in a proteinase K buffer (500 mM NaCl, 10 mM Tris-HCl, pH 7.2, 1.5 mM MgCl2, 10 mM EDTA, 2% SDS and 0.5 mg/ml of proteinase K) for 45 min at 55 C. Total RNAs were isolated using TriReagent according to the manufacturer's protocol. One microgram of RNA was reverse transcribed using M-MLV first-strand synthesis system (Life Technologies) according to the manufacturer's instructions in a total of 20 μL. One microliter of cDNA preparation was subsequently used in a semi-quantitative PCR analysis according to standard protocol (ReddyMix, Thermo Scientific). Primers are shown in the following table 2:

TABLE 2

| Primer | SEQ ID | Species/Gene/ | Sequence |
|---|---|---|---|
| Name | NO. | Exon | (5'-3') |
| Mbnl1.F | 35 | Mouse-Human/ | GCTGCCCAATA |
| | | mbnl1/exon5 | CCAGGTCAAC |
| Mbnl1.R | 36 | Mouse-Human/ | TGGTGGGAGAA |
| | | mbnl1/exon5 | ATGCTGTATGC |
| Clcn1.F | 37 | Mouse/clcn1/ | TTCACATCGCC |
| | | exon7a | AGCATCTGTGC |
| Clcn1.R | 38 | Mouse/clcn1/ | CACGGAACACA |
| | | exon7a | AAGGCACTGAA |
| | | | TGT |

PCR amplification was carried out for 25-35 cycles within the linear range of amplification for each gene. PCR products were resolved on 1.5-2% agarose gels, ethidium bromide-stained and quantified with ImageJ software. The ratios of exon inclusion were quantified as a percentage of inclusion relative to total intensity of isoform signals. To quantify the mRNA expression, real-time PCR was performed according to the manufacturer's instructions. PCR cycles were a 15-min denaturation step followed by 50 cycles with a 94 C denaturation for 15 s, 58 C annealing for 20 s and 72 C extension for 20 s.

### Fluorescent In Situ Hybridization/Immunofluorescence

Fluorescent in situ hybridization (FISH) experiments were done as previously described (6) using a Cy3-labeled 2′OMe (CAG) 7 probe (Eurogentec). For combined FISH-Immunofluorescence experiments, immunofluorescence staining was done after FISH last washing with a rabbit polyclonal anti-MBNL1 antibody followed by a secondary Alexa Fluor 488-conjugated goat anti-rabbit (1:500, Life technologies) antibody.

### ELISA Based Measurements of Oligonucleotide Concentrations in Tissues

Customized Hybridization-Based ELISAs were developed to determine the concentration of PMO oligonucleotides using phosphorothioate probes having phosphorothioate linkages (Sequence (5′->3′) [DIG]C*T*G*C*T*G*C*TGCTGCT*G*C*T*G*C*T*G [BIO](SEQ ID NO:39); * represents a phosphorothioate bond) double-labelled with digoxigenin and biotin. The assay had a linear detection range of 5-250 pM (R2>0.99) in mouse serum and tissue lysates. The probe was used to detect peptide-PMOs or naked PMO concentrations in eight different tissues (brain, kidney, liver, lung, heart, diaphragm, gastrocnemius and quadriceps) from treated HSA-LR mice.

### Results

In this work, the inventors used lysine-rich cell-penetrating peptides having specific structure and showed that such a peptide conjugated to a [CAG]₇ morpholino phosphorodiamidate oligomer (PMO) dramatically enhanced ASO delivery into skeletal and cardiac muscles of DM1 model HSA-LR mice following systemic administration in comparison to the unconjugated PMO and other peptide carrier conjugate strategies. Thus, low dose treatment of a conjugate formed of peptide-[CAG]₇ PMO as claimed herein targeting pathologic expansions was sufficient to reverse both splicing defects and myotonia in DM1 mice (HSALR). Moreover, treated DM1 patient derived muscle cells (myoblasts) showed that the peptide[CAG]₇ PMO conjugates as claimed herein specifically target mutant CUGexp-DMPK transcripts to abrogate the detrimental sequestration of MBNL1 splicing factor by nuclear RNA foci and consequently MBNL1 functional loss, responsible for splicing defects and muscle dysfunction. Our results demonstrate that the peptide-[CAG]₇ PMO conjugates as claimed herein induce high efficacy correction of DM1-associated phenotypes at both molecular and functional levels, and strongly support the use of these peptide-conjugates for systemic corrective therapy in DM1.

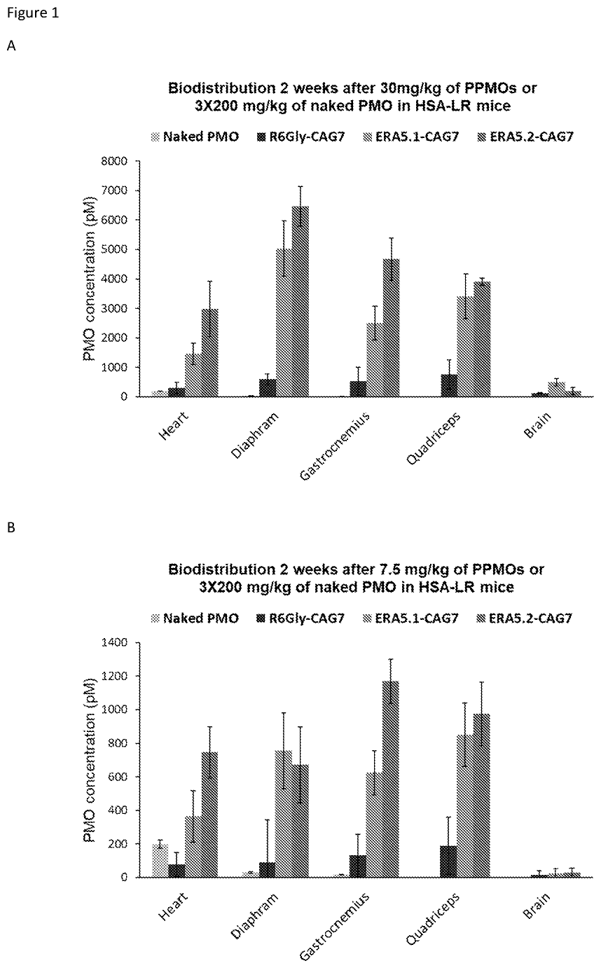

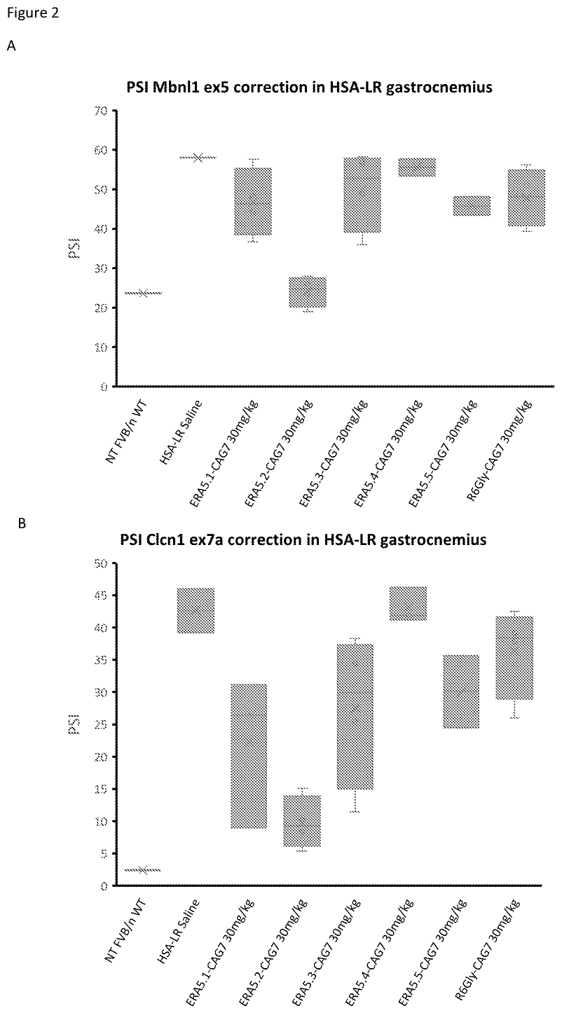

The inventors have produced data with conjugates comprising peptide carriers which contain no R residues, that have wider therapeutic window and safer toxicology profile than previous cell penetrating peptides and, therefore, constitute more promising candidates to be tested in DM1 patients. These new generation of so called ERA5 peptides have shown high efficacy delivering the antisense cargo (CAG7-PMO) to hard to reach tissues such as cardiac and skeletal muscle (FIG. 1). Biodistribution of naked PMO versus conjugates formed with carrier peptides ERA5.1, ERA5.2 and the benchmark R6Gly was assessed by ELISA to quantify delivery of peptide-[CAG]₇ PMO conjugate. Detection of PMO in critically affected tissues in DM1, such as skeletal muscle and heart, is important for drug delivery development. A single intravenous injection of peptide-[CAG]₇ PMO conjugate at 30 mg/kg or 3 injections at 200 mg/kg of naked PMO were administered to HSA-LR mice (total 600 mg/kg). Gastrocnemius, quadriceps, diaphragm, heart and brain were analysed for PMO detection 2 weeks post administration. The unconjugated naked [CAG]₇ PMO has low to non-detectable levels in all tissues tested, however the [CAG]₇ PMO conjugated to peptide carriers ERA5.1 and ERA5.2 was detected at higher levels than the benchmark peptide R6Gly. In general peptide-[CAG]₇ PMO conjugates were detected in heart, quadriceps, gastrocnemius and diaphragm at 1 nM-7 nM 2 weeks after 30 mg/kg injections (FIGS. 1, A) and at 0.3 to 1.2 nM after 7.5 m/kg treatments (FIG. 2, B).

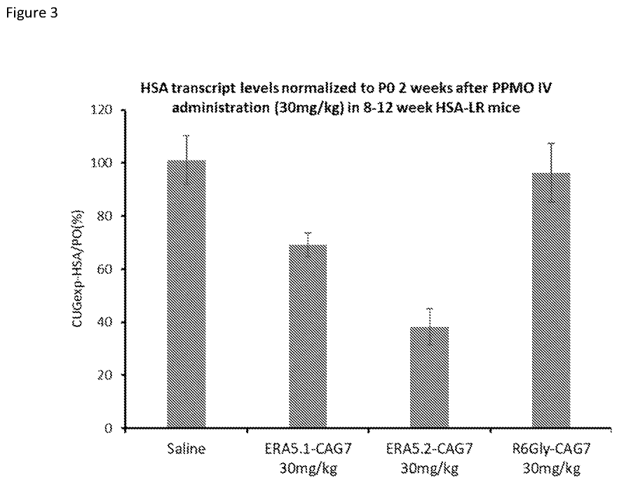

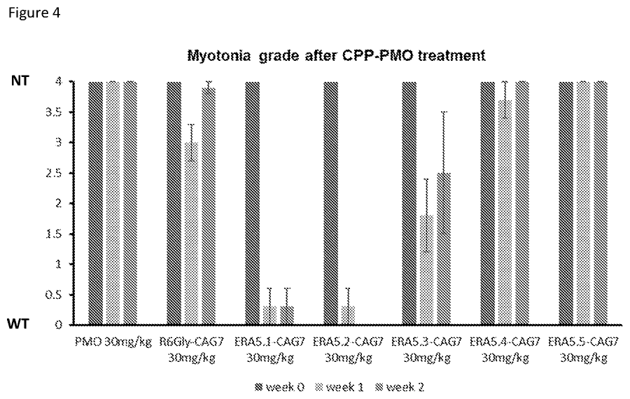

The inventors tested if these new peptides were also active to correct myotonia and splicing changes in HSA-LR mice. To do so the leading peptide carriers of the ERA5 series were compared with a benchmark peptide carrier R6Gly. the inventors were able to show that conjugates comprising ERA5 peptides and the antisense cargo (CAG7 PMO) correct splicing defects in muscle when they are administered systemically (IV, tail vein) (FIG. 2). Thus, the conjugates of the invention correct 50-90% of the mis-splicing in Clcn1 (ex7a) and Mbnl1 (ex5) in HSA-LR gastrocnemius two weeks after treatment in 8-12-week HSA-LR mice (FIG. 2). These treatments also reduce the HSA transcript levels containing the toxic repetitive sequence when normalized to P0 2 weeks after PPMO IV administration (30 mg/kg) (FIG. 3). These molecular corrections are associated to the reversal of DM1 pathology. For example, FIG. 4 shows how myotonia is corrected to wild type levels 1 week after administration (30 mg/kg) of conjugates formed with ERA5.1 and ERA5.2 conjugates. Importantly, pathology reversal is not associated to changes in urine toxicity biomarkers (kidney toxicity, Kim-1 levels). After administration of conjugates formed with ERA5.1, ERA5.2, ERA5.3, ERA5.4 and

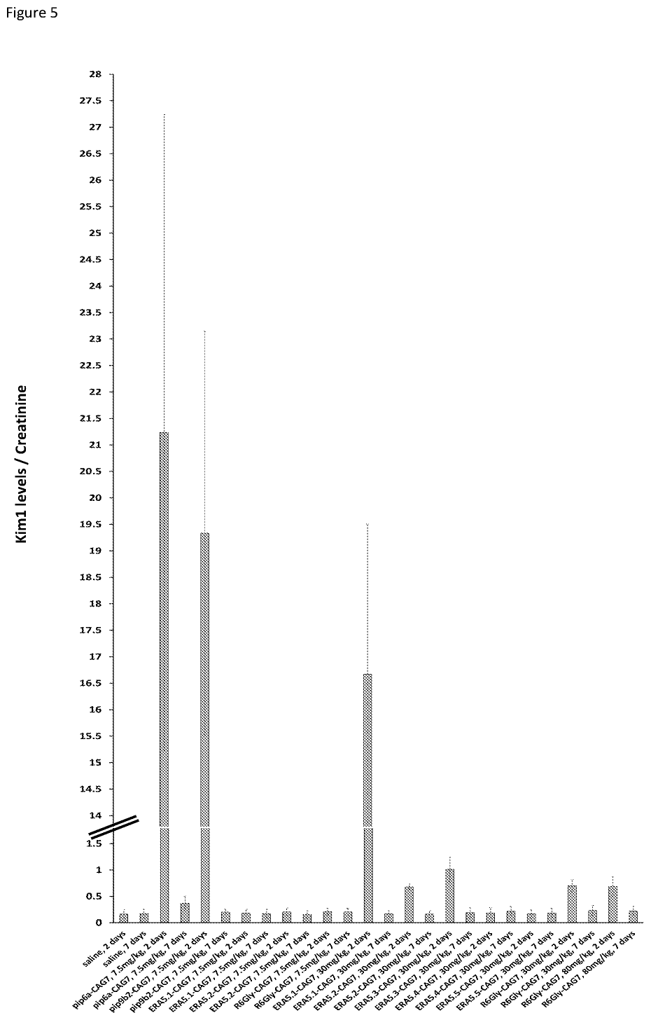

ERA5.5 Kim-1 levels were similar to saline control injections, in contrast to the fold increases typically induced by the equivalent R substituted carriers (pip6a and pip9b2) 2 days even after lower doses, 7.5 mg/kg IV administration (FIG. 5). Notably, conjugates formed with prior peptide carriers such as Pip6a-[CAG]7 PMO or Pip9b2-[CAG]7 cannot be tested at >20 mg/kg without causing high rates of mortality in mice, this is contrary to the conjugates of the invention for which the concentration can be increased more than 5-fold without causing any mortality.

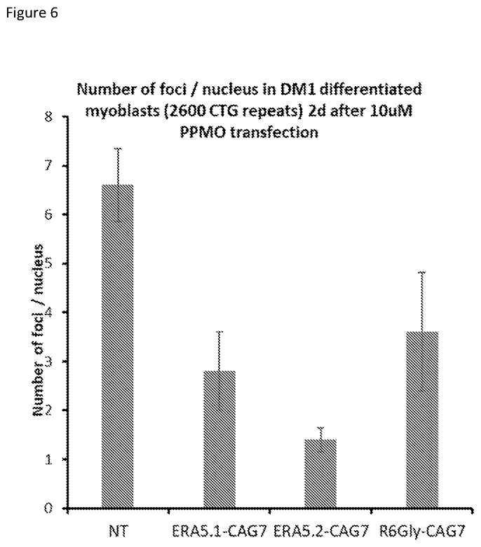

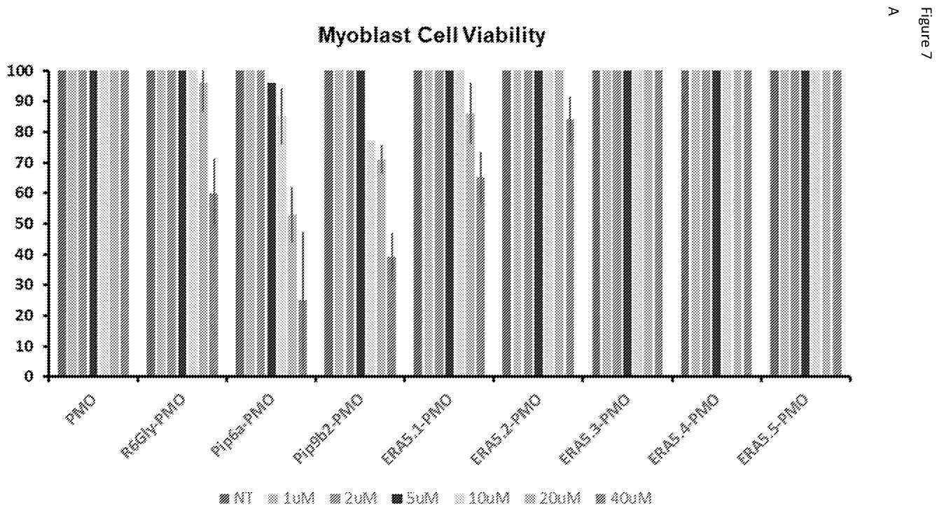

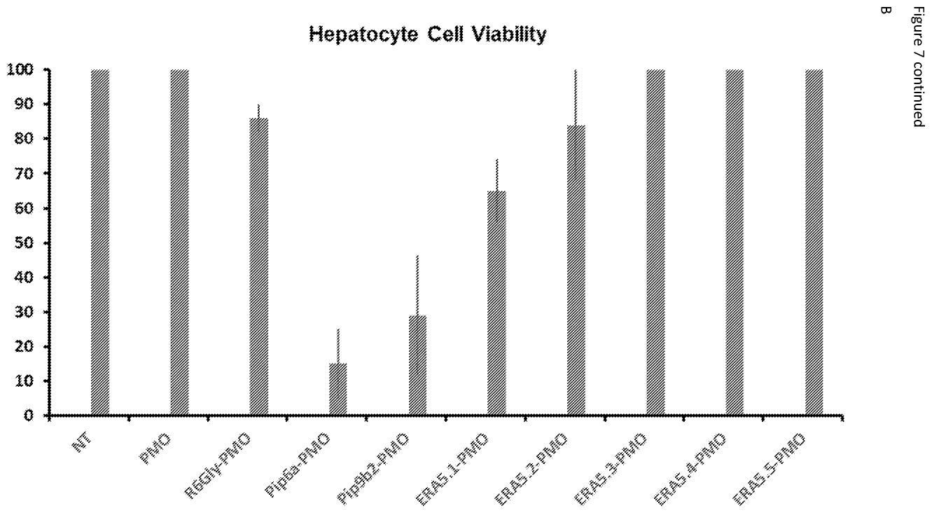

Conjugates formed with carrier peptides of the ERA5 series reduce the number of foci comprising mutant DMPK RNA per nucleus in DM1 differentiated myoblasts (2600 CTG repeats) 2 d after 10 uM PPMO transfection (FIG. 6). None of the concentrations tested caused reductions of cell viability in human hepatocytes (1-40 μM) contrary to a similar comparative conjugate formed from a known ‘Pip’ carrier peptides; Pip6a-PMO and Pip9b2-PMO that induced significant cell mortality (>50%) at 40 μM (FIG. 7). Many of the concentrations tested caused no reduction in cell viability of human myoblasts, and fared better compared to similar comparative conjugates formed from known ‘Pip’ carrier peptides Pip6a-PMO and Pip9b2-PMO that induced cell mortality at lower doses (FIG. 7).

With this preliminary data the inventors have shown that conjugates formed from ERA5 peptides with a [CAG]7 PMO are more active than the benchmark conjugate R6Gly-CAG7 (FIGS. 1, 2, 3 and 4) and as active have wider therapeutic window than the PIP series (FIG. 5). The efficacy and toxicology data indicate that conjugates formed with carrier peptides of the ERA5 series as claimed are especially active blocking the sequestration of MBNL1 by the expanded CTG repeats in individuals affected by DM1, and induce low toxicity. These conjugates are able to correct the DM1 phenotype. These new conjugates further have wider therapeutic windows than conjugates formed with previous peptide carriers and, therefore, they are closer to realisation in the clinic.

In summary, the inventors show strong evidence supporting (1) that peptide-[CAG]₇ PMO block the pathological interactions of MBNL1 with the nuclear mutant CUGexp-RNA and rescue the downstream effects on RNA-splicing; (2) that the peptide conjugated antisense oligonucleotide approach allows the treatment to be delivered to inaccessible tissues like heart in diaphragm; (3) that the strong effect of the [CAG]₇ PMO directly targeting the disease mutation combined with the ability of the peptide carrier technology to deliver the treatment in vivo with high efficacy converges on the powerful reversal of the DM1 phenotype in skeletal muscle DM1 mice (HSA-LR) to wild type levels. These pieces of evidence strongly suggest that peptide-[CAG]₇ conjugates are likely to have a strong disease modifying effect in DM1. In fact, the experiments show that the effect that we observe in the HSA-LR mice is not only preventing the worsening of the DM1 pathology but that it is causing reversal of the disease phenotype. The expanded CUG-transcripts are already expressed in pups, and HSA-LR mice have significant myotonia present by the age of 1 month. The animals used to generate the results supporting this application were treated at the age of at least 2 months and even 7 months, well beyond the point at which the molecular and functional phenotype of DM1 develops.

### CONCLUSIONS

Conjugates comprising ERA5 carrier peptides and a [CAG]₇ PMO showed positive biodistribution evaluation revealed optimal delivery to critically affected tissues in DM1 such as skeletal and cardiac muscle.

Conjugates comprising ERA5 carrier peptides and a [CAG]₇ PMO (10 μM) are able to reduce >50% the number of nuclear foci (at doses that did not decreased cell viability) in DM1 patient myoblasts and controls. None of the concentrations tested caused reductions of cell viability (1-40 μM) contrary to comparative conjugates formed with other carrier peptides (PIP series) that induced significant cell mortality (>50%) at 20 μM or higher concentrations.

Conjugates comprising ERA5 carrier peptides (lysine rich) and a [CAG]₇ PMO induced splicing corrections of 30%-90% in Clcn1 exon 7a and Mbnl1 exon 5 at 30 mg/kg (IV) and that dose of ERA5 conjugates is associated with less toxicity than 7.5 mg/kg of comparative conjugates formed with other carrier peptides (PIP series, arginine rich) in HSA-LR mice.

ERA5 [CAG]₇ PMO conjugates are more potent than the benchmark conjugate R6gly [CAG]7 PMO correcting splicing and decreasing myotonia to wild type levels 1 weeks after a single injection at 30 mg/kg (IV).

Urine biochemistry tests for kidney function show no changes in comparison with saline in HSA-LR or wild type mice and mild changes in HSA-LR Kim1 levels after=>30 mg/kg (IV) treatments comprising ERA5 carrier peptides and a [CAG]7 PMO of comparative conjugates formed with other carrier peptides (PIP series) at 7.5 mg/kg.

Sequences
(SEQ ID NO: 1)
KXKKBKK
(SEQ ID NO: 2)
KXKKBKKXK
(SEQ ID NO: 3)
KBKKBKK
(SEQ ID NO: 4)
KBKKBK
(SEQ ID NO: 5)
KBKK
(SEQ ID NO: 6)
KXKBKXK
(SEQ ID NO: 7)
KBKXK
(SEQ ID NO: 8)
KBKBK
(SEQ ID NO: 9)
BKBK
(SEQ ID NO: 10)
YQFLI
(SEQ ID NO: 11)
ILFQY
(SEQ ID NO: 12)
YRLFI
(SEQ ID NO: 13)
FQILY
(SEQ ID NO: 14)
KXKKBKK FQILY KBKXK (ERA5.2)
(SEQ ID NO: 15)
KXKKBKKXK YQFLI KXKBKXK (ERA5.1)
(SEQ ID NO: 16)
KBKKBKK FQILY KBKXK
(SEQ ID NO: 17)
KBKKBKK FQILY KBKBK (ERA5.3)
(SEQ ID NO: 18)
KBKK YQFLI KBKXK (ERA5.4)
(SEQ ID NO: 19)
KBKKBK FQILY BKBK (ERA5.5)
(SEQ ID NO. 20)
KXKKBKK FQILY KBKXK-C
(SEQ ID NO. 21)
KXKKBKKXK YQFLI KXKBKXK-C
(SEQ ID NO. 22)
KBKKBKK FQILY KBKXK-C
(SEQ ID NO. 23)
KBKKBKK FQILY KBKBK-C
(SEQ ID NO. 24)
KBKK YQFLI KBKXK-C
(SEQ ID NO. 25)
KBKKBK FQILY BKBK-C
(SEQ ID NO. 26)
CAGCAGCAGCAGCAGCAGCAG
(SEQ ID NO. 27)
Ac-KXKKBKKXK YQFLI KXKBKXK-C (ERA5.1)
(SEQ ID NO. 28)
Ac-KBKKBKK FQILY KBKXK-C
(SEQ ID NO. 41)
Ac-KXKKBKK FQILY KBKXK-C (ERA5.2)
(SEQ ID NO. 29)
Ac-KBKKBKK FQILY KBKBK-C (ERA5.3)
(SEQ ID NO. 30)
AC-KBKK YQFLI KBKXK-C (ERA5.4)
(SEQ ID NO. 31)
Ac-KBKKBK FQILY BKBK-C (ERA5.5)
(SEQ ID NO. 32)
Ac-RRRRRR-Gly
(SEQ ID NO. 33)
Ac-RXRRBRRXR-YQFLI-RXRBRXR-C
(SEQ ID NO. 34)
Ac-RXRRBRR-FQILY-RBRXR-C
(SEQ ID NO. 35)
GCTGCCCAATACCAGGTCAAC
(SEQ ID NO. 36)
TGGTGGGAGAAATGCTGTATGC
(SEQ ID NO. 37)
TTCACATCGCCAGCATCTGTGC
(SEQ ID NO. 38)
CACGGAACACAAAGGCACTGAATGT
(SEQ ID NO. 39)
[DIG]C*T*G*C*T*G*C*TGCTGCT*G*
C*T*G*C*T*G[BIO]
(SEQ ID NO. 40)
RRRRRR

1. A peptide having a total length of 40 amino acid residues or less, the peptide comprising at least two cationic domains and at least one hydrophobic domain, wherein the at least two cationic domains each comprise a plurality of lysine residues and wherein the peptide does not contain arginine.

2. A peptide according to claim 1 wherein the at least two cationic domains each comprise cationic amino acid residues, preferably the at least two cationic domains each comprise at least 40%, at least 50%, at least 60%, at least 70%, at least 80%, at least 90% cationic amino acid residues, more preferably wherein the cationic amino acid residues are selected from lysine or histidine.

3. A peptide according to claim 1 or 2, wherein the at least two cationic domains comprise one or more amino acid residues selected from lysine, aminohexanoic acid, and beta-alanine, preferably wherein the at least two cationic domains consist of amino acid residues selected from lysine, aminohexanoic acid, and beta-alanine.

4. A peptide according to any of claims 1-3 wherein the at least two cationic domains each comprise a majority of lysine residues, preferably wherein the at least two cationic domains each comprise at least 60%, at least 70%, at least 80%, at least 90% lysine residues.

5. A peptide according to any of claims 1-4 wherein the at least two cationic domains each comprise between 2-10 lysine residues, preferably between 2-6 lysine residues.

6. A peptide according to any of claims 1-5 wherein the peptide comprises two cationic domains, preferably wherein each cationic domain comprises one of the following sequences: KXKKBKK (SEQ ID NO. 1), KXKKBKKXK (SEQ ID NO. 2), KBKKBKK (SEQ ID NO. 3), KBKKBK (SEQ ID NO. 4), KBKK (SEQ ID NO. 5), KXKBKXK (SEQ ID NO. 6), KBKXK (SEQ ID NO. 7), KBKBK (SEQ ID NO. 8), and BKBK (SEQ ID NO. 9), more preferably wherein each cationic domain consists of one of the following sequences: KXKKBKK (SEQ ID NO. 1), KXKKBKKXK (SEQ ID NO. 2), KBKKBKK (SEQ ID NO. 3), KBKKBK (SEQ ID NO. 4), KBKK (SEQ ID NO. 5), KXKBKXK (SEQ ID NO. 6), KBKXK (SEQ ID NO. 7), KBKBK (SEQ ID NO. 8), and BKBK (SEQ ID NO. 9).

7. A peptide according to any preceding claim, wherein the at least one hydrophobic domain comprises hydrophobic amino acid residues, preferably at least 40%, at least 50%, at least 60%, at least 70%, at least 80%, at least 90% hydrophobic amino acid residues, more preferably the at least one hydrophobic domain consists of hydrophobic amino acid residues.

8. A peptide according to any preceding claim wherein the peptide comprises one hydrophobic domain, preferably wherein the hydrophobic domain comprises one of the following sequences: YQFLI (SEQ ID NO:10), ILFQY (SEQ ID NO:11), YRLFI (SEQ ID NO: 12), and FQILY (SEQ ID NO:13), more preferably wherein the hydrophobic domain consists of one of the following sequences: YQFLI (SEQ ID NO:10), ILFQY (SEQ ID NO:11), YRLFI (SEQ ID NO:12), and FQILY (SEQ ID NO:13).

9. A peptide according to any preceding claim wherein the at least one hydrophobic domain is flanked by the at least two cationic domains, preferably wherein the peptide comprises the structure: [first cationic domain]-[hydrophobic domain]-[second cationic domain], more preferably wherein the peptide consists of the structure [first cationic domain]-[hydrophobic domain]-[second cationic domain].

10. A peptide according to any preceding claim wherein the peptide consists of two cationic domains and one hydrophobic domain.

11. A peptide according to claim 10, wherein the two cationic arm domains consist of a first cationic arm domain selected from: KXKKBKK (SEQ ID NO. 1), KXKKBKKXK (SEQ ID NO. 2), KBKKBKK (SEQ ID NO. 3), KBKKBK (SEQ ID NO. 4), and KBKK (SEQ ID NO. 5), and a second cationic arm domain selected from: KXKBKXK (SEQ ID NO. 6), KBKXK (SEQ ID NO. 7), KBKBK (SEQ ID NO. 8), and BKBK (SEQ ID NO. 9).

12. A peptide according to claim 10 or 11 wherein the hydrophobic domain is selected from: YQFLI (SEQ ID NO: 10) or FQILY (SEQ ID NO:13).

13. A peptide according to any preceding claim, wherein the peptides is less than 35 amino acid residues in length, preferably less than 30 amino acid residues in length, preferably less than 25 amino acid residues in length, preferably less than 20 amino acid residues in length.

14. A peptide according to any preceding claim wherein the peptide comprises or consists of one of the following sequences:
(SEQ ID NO: 14)
KXKKBKK FQILY KBKXK (ERA 5.2)
(SEQ ID NO: 15)
KXKKBKKXK YQFLI KXKBKXK (ERA 5.1)
(SEQ ID NO: 16)
KBKKBKK FQILY KBKXK
(SEQ ID NO: 17)
KBKKBKK FQILY KBKBK (ERA 5.3)
(SEQ ID NO: 18)
KBKK YQFLI KBKXK (ERA 5.4)
(SEQ ID NO: 19)
KBKKBK FQILY BKBK (ERA 5.5)

15. A conjugate comprising a peptide according to any preceding claim, covalently linked to a therapeutic molecule.

16. A conjugate according to claim 15, wherein the peptide is covalently linked by a linker, preferably the linker is selected from: G, BC, XC, C, GGC, BBC, BXC, XBC, X, XX, B, BB, BX and XB, preferably wherein the linker is C.

17. A conjugate according to any of claim 15 or 16, wherein the therapeutic molecule is selected from a nucleic acid, peptide nucleic acid, antisense oligonucleotide, short interfering RNA, micro RNA, peptide, cyclic peptide, protein, pharmaceutical and drug, preferably the therapeutic molecule is an antisense oligonucleotide, more preferably wherein the antisense oligonucleotide is a PMO.

18. A pharmaceutical composition comprising the conjugate according any of claims 15-17.

19. A conjugate according to any of claims 15-17, or pharmaceutical composition according to claim 19 for use as a medicament.

20. A conjugate according to any of claims 15-17, or pharmaceutical composition according to claim 19 for use in the treatment of diseases of the neuromuscular system or musculoskeletal system, preferably genetic diseases of the neuromuscular system or musculoskeletal system, more preferably hereditary genetic diseases of the neuromuscular system or musculoskeletal system.


SEQUENCE LISTING (41 sequences)

SEQ ID NO: 1 - protein, 7 aa, synthetic construct
  SITE 2: note = X denotes any form of the artificial, synthetically produced amino acid aminohexanoic acid, preferably 6-aminohexanoic acid
  SITE 5: note = bAla
KXKKAKK

SEQ ID NO: 2 - protein, 9 aa, synthetic construct
  SITE 2: note = X denotes any form of the artificial, synthetically produced amino acid aminohexanoic acid, preferably 6-aminohexanoic acid
  SITE 8: note = X denotes any form of the artificial, synthetically produced amino acid aminohexanoic acid, preferably 6-aminohexanoic acid
  SITE 5: note = bAla
KXKKAKKXK

SEQ ID NO: 3 - protein, 7 aa, synthetic construct
  SITE 2: note = bAla
  SITE 5: note = bAla
KAKKAKK

SEQ ID NO: 4 - protein, 6 aa, synthetic construct
  SITE 2: note = bAla
  SITE 5: note = bAla
KAKKAK

SEQ ID NO: 5 - protein, 4 aa, synthetic construct
  SITE 2: note = bAla
KAKK

SEQ ID NO: 6 - protein, 7 aa, synthetic construct
  SITE 2: note = X denotes any form of the artificial, synthetically produced amino acid aminohexanoic acid, preferably 6-aminohexanoic acid
  SITE 6: note = X denotes any form of the artificial, synthetically produced amino acid aminohexanoic acid, preferably 6-aminohexanoic acid
  SITE 4: note = bAla
KXKAKXK

SEQ ID NO: 7 - protein, 5 aa, synthetic construct
  SITE 4: note = X denotes any form of the artificial, synthetically produced amino acid aminohexanoic acid, preferably 6-aminohexanoic acid
  SITE 2: note = bAla
KAKXK

SEQ ID NO: 8 - protein, 5 aa, synthetic construct
  SITE 2: note = bAla
  SITE 4: note = bAla
KAKAK

SEQ ID NO: 9 - protein, 4 aa, synthetic construct
  SITE 1: note = bAla
  SITE 3: note = bAla
AKAK

SEQ ID NO: 10 - protein, 5 aa, synthetic construct
YQFLI

SEQ ID NO: 11 - protein, 5 aa, synthetic construct
ILFQY

SEQ ID NO: 12 - protein, 5 aa, synthetic construct
YRLFI

SEQ ID NO: 13 - protein, 5 aa, synthetic construct
FQILY

SEQ ID NO: 14 - protein, 17 aa, synthetic construct
  SITE 2: note = X denotes any form of the artificial, synthetically produced amino acid aminohexanoic acid, preferably 6-aminohexanoic acid
  SITE 16: note = X denotes any form of the artificial, synthetically produced amino acid aminohexanoic acid, preferably 6-aminohexanoic acid
  SITE 5: note = bAla
  SITE 14: note = bAla
KXKKAKKFQILYKAKXK

SEQ ID NO: 15 - protein, 21 aa, synthetic construct
  SITE 2: note = X denotes any form of the artificial, synthetically produced amino acid aminohexanoic acid, preferably 6-aminohexanoic acid
  SITE 8: note = X denotes any form of the artificial, synthetically produced amino acid aminohexanoic acid, preferably 6-aminohexanoic acid
  SITE 16: note = X denotes any form of the artificial, synthetically produced amino acid aminohexanoic acid, preferably 6-aminohexanoic acid
  SITE 20: note = X denotes any form of the artificial, synthetically produced amino acid aminohexanoic acid, preferably 6-aminohexanoic acid
  SITE 5: note = bAla
  SITE 18: note = bAla
KXKKAKKXKYQFLIKXKAKXK

SEQ ID NO: 16 - protein, 17 aa, synthetic construct
  SITE 16: note = X denotes any form of the artificial, synthetically produced amino acid aminohexanoic acid, preferably 6-aminohexanoic acid
  SITE 2: note = bAla
  SITE 5: note = bAla
  SITE 14: note = bAla
KAKKAKKFQILYKAKXK

SEQ ID NO: 17 - protein, 17 aa, synthetic construct
  SITE 2: note = bAla
  SITE 5: note = bAla
  SITE 14: note = bAla
  SITE 16: note = bAla
KAKKAKKFQILYKAKAK

SEQ ID NO: 18 - protein, 14 aa, synthetic construct
  SITE 13: note = X denotes any form of the artificial, synthetically produced amino acid aminohexanoic acid, preferably 6-aminohexanoic acid
  SITE 2: note = bAla
  SITE 11: note = bAla
KAKKYQFLIKAKXK

SEQ ID NO: 19 - protein, 15 aa, synthetic construct
  SITE 2: note = bAla
  SITE 5: note = bAla
  SITE 12: note = bAla
  SITE 14: note = bAla
KAKKAKFQILYAKAK

SEQ ID NO: 20 - protein, 18 aa, synthetic construct
  SITE 2: note = X denotes any form of the artificial, synthetically produced amino acid aminohexanoic acid, preferably 6-aminohexanoic acid
  SITE 16: note = X denotes any form of the artificial, synthetically produced amino acid aminohexanoic acid, preferably 6-aminohexanoic acid
  SITE 5: note = bAla
  SITE 14: note = bAla
KXKKAKKFQILYKAKXKC

SEQ ID NO: 21 - protein, 22 aa, synthetic construct
  SITE 2: note = X denotes any form of the artificial, synthetically produced amino acid aminohexanoic acid, preferably 6-aminohexanoic acid
  SITE 8: note = X denotes any form of the artificial, synthetically produced amino acid aminohexanoic acid, preferably 6-aminohexanoic acid
  SITE 16: note = X denotes any form of the artificial, synthetically produced amino acid aminohexanoic acid, preferably 6-aminohexanoic acid
  SITE 20: note = X denotes any form of the artificial, synthetically produced amino acid aminohexanoic acid, preferably 6-aminohexanoic acid
  SITE 5: note = bAla
  SITE 18: note = bAla
KXKKAKKXKYQFLIKXKAKXKC

SEQ ID NO: 22 - protein, 18 aa, synthetic construct
  SITE 16: note = X denotes any form of the artificial, synthetically produced amino acid aminohexanoic acid, preferably 6-aminohexanoic acid
  SITE 2: note = bAla
  SITE 5: note = bAla
  SITE 14: note = bAla
KAKKAKKFQILYKAKXKC

SEQ ID NO: 23 - protein, 18 aa, synthetic construct
  SITE 2: note = bAla
  SITE 5: note = bAla
  SITE 14: note = bAla
  SITE 16: note = bAla
KAKKAKKFQILYKAKAKC

SEQ ID NO: 24 - protein, 15 aa, synthetic construct
  SITE 13: note = X denotes any form of the artificial, synthetically produced amino acid aminohexanoic acid, preferably 6-aminohexanoic acid
  SITE 2: note = bAla
  SITE 11: note = bAla
KAKKYQFLIKAKXKC

SEQ ID NO: 25 - protein, 16 aa, synthetic construct
  SITE 2: note = bAla
  SITE 5: note = bAla
  SITE 12: note = bAla
  SITE 14: note = bAla
KAKKAKFQILYAKAKC

SEQ ID NO: 26 - DNA, 21 bp, synthetic construct
  source 1..21: mol_type = other DNA
cagcagcagcagcagcagcag

SEQ ID NO: 27 - protein, 22 aa, synthetic construct
  SITE 2: note = X denotes any form of the artificial, synthetically produced amino acid aminohexanoic acid, preferably 6-aminohexanoic acid
  SITE 8: note = X denotes any form of the artificial, synthetically produced amino acid aminohexanoic acid, preferably 6-aminohexanoic acid
  SITE 16: note = X denotes any form of the artificial, synthetically produced amino acid aminohexanoic acid, preferably 6-aminohexanoic acid
  SITE 20: note = X denotes any form of the artificial, synthetically produced amino acid aminohexanoic acid, preferably 6-aminohexanoic acid
  SITE 1: note = N-acetylation
  SITE 5: note = bAla
  SITE 18: note = bAla
KXKKAKKXKYQFLIKXKAKXKC

SEQ ID NO: 28 - protein, 18 aa, synthetic construct
  SITE 16: note = X denotes any form of the artificial, synthetically produced amino acid aminohexanoic acid, preferably 6-aminohexanoic acid
  SITE 1: note = N-acetylation
  SITE 2: note = bAla
  SITE 5: note = bAla
  SITE 14: note = bAla
KAKKAKKFQILYKAKXKC

SEQ ID NO: 29 - protein, 18 aa, synthetic construct
  SITE 1: note = N-acetylation
  SITE 2: note = bAla
  SITE 5: note = bAla
  SITE 14: note = bAla
  SITE 16: note = bAla
KAKKAKKFQILYKAKAKC

SEQ ID NO: 30 - protein, 15 aa, synthetic construct
  SITE 13: note = X denotes any form of the artificial, synthetically produced amino acid aminohexanoic acid, preferably 6-aminohexanoic acid
  SITE 1: note = N-acetylation
  SITE 2: note = bAla
  SITE 11: note = bAla
KAKKYQFLIKAKXKC

SEQ ID NO: 31 - protein, 16 aa, synthetic construct
  SITE 1: note = N-acetylation
  SITE 2: note = bAla
  SITE 5: note = bAla
  SITE 12: note = bAla
  SITE 14: note = bAla
KAKKAKFQILYAKAKC

SEQ ID NO: 32 - protein, 7 aa, synthetic construct
  SITE 1: note = N-acetylation
RRRRRRG

SEQ ID NO: 33 - protein, 22 aa, synthetic construct
  SITE 2: note = X denotes any form of the artificial, synthetically produced amino acid aminohexanoic acid, preferably 6-aminohexanoic acid
  SITE 8: note = X denotes any form of the artificial, synthetically produced amino acid aminohexanoic acid, preferably 6-aminohexanoic acid
  SITE 16: note = X denotes any form of the artificial, synthetically produced amino acid aminohexanoic acid, preferably 6-aminohexanoic acid
  SITE 20: note = X denotes any form of the artificial, synthetically produced amino acid aminohexanoic acid, preferably 6-aminohexanoic acid
  SITE 1: note = N-acetylation
  SITE 5: note = bAla
  SITE 18: note = bAla
RXRRARRXRYQFLIRXRARXRC

SEQ ID NO: 34 - protein, 18 aa, synthetic construct
  SITE 2: note = X denotes any form of the artificial, synthetically produced amino acid aminohexanoic acid, preferably 6-aminohexanoic acid
  SITE 16: note = X denotes any form of the artificial, synthetically produced amino acid aminohexanoic acid, preferably 6-aminohexanoic acid
  SITE 1: note = N-acetylation
  SITE 5: note = bAla
  SITE 14: note = bAla
RXRRARRFQILYRARXRC

SEQ ID NO: 35 - DNA, 21 bp, synthetic construct
  source 1..21: mol_type = other DNA; note = Primer name Mbnl1.F
gctgcccaataccaggtcaac

SEQ ID NO: 36 - DNA, 22 bp, synthetic construct
  source 1..22: mol_type = other DNA; note = Primer name Mbnl1.R
tggtgggagaaatgctgtatgc

SEQ ID NO: 37 - DNA, 22 bp, synthetic construct
  source 1..22: mol_type = other DNA; note = Primer name Clcn1.F
ttcacatcgccagcatctgtgc

SEQ ID NO: 38 - DNA, 25 bp, synthetic construct
  source 1..25: mol_type = other DNA; note = Primer name Clcn1.R
cacggaacacaaaggcactgaatgt

SEQ ID NO: 39 - DNA, 21 bp, synthetic construct
  source 1..21: mol_type = other DNA
  misc_difference 1: note = Digoxigenin labelled nucleotide
  misc_difference 21: note = Biotin labelled nucleotide
  misc_difference 1^2: note = Phosphorothioate linkage
  misc_difference 2^3: note = Phosphorothioate linkage
  misc_difference 3^4: note = Phosphorothioate linkage
  misc_difference 4^5: note = Phosphorothioate linkage
  misc_difference 5^6: note = Phosphorothioate linkage
  misc_difference 6^7: note = Phosphorothioate linkage
  misc_difference 7^8: note = Phosphorothioate linkage
  misc_difference 14^15: note = Phosphorothioate linkage
  misc_difference 15^16: note = Phosphorothioate linkage
  misc_difference 16^17: note = Phosphorothioate linkage
  misc_difference 17^18: note = Phosphorothioate linkage
  misc_difference 18^19: note = Phosphorothioate linkage
  misc_difference 19^20: note = Phosphorothioate linkage
  misc_difference 20^21: note = Phosphorothioate linkage
ctgctgctgctgctgctgctg

SEQ ID NO: 40 - protein, 6 aa, synthetic construct
RRRRRR

SEQ ID NO: 41 - protein, 18 aa, synthetic construct
  SITE 2: note = X denotes any form of the artificial, synthetically produced amino acid aminohexanoic acid, preferably 6-aminohexanoic acid
  SITE 16: note = X denotes any form of the artificial, synthetically produced amino acid aminohexanoic acid, preferably 6-aminohexanoic acid
  SITE 1: note = N-acetylation
  SITE 5: note = bAla
  SITE 14: note = bAla
KXKKAKKFQILYKAKXKC